(19) United States
(12) Patent Application Publication
(10) Pub. No.: US 2015/0023920 A1
(43) Pub. Date: Jan. 22, 2015

(54) NOVEL COMPOSITIONS AND METHODS FOR PREVENTING OR TREATING CANCER METASTASIS

(71) Applicant: Drexel University, Philadelphia, PA (US)

(72) Inventors: Alessandro Fatatis, Penn Valley, PA (US); Michael Russell, Lansdowne, PA (US); Qingxin (Cindy) Lui, Philadelphia, PA (US)

(73) Assignee: Drexel University, Philadslphia, PA (US)

(21) Appl. No.: 14/389,086

(22) PCT Filed: Mar. 13, 2013
(86) PCT No.: PCT/US2013/030906

§ 371 Date: Sep. 29, 2014

Related U.S. Application Data
(60) Provisional application No. 61/617,447, filed on Mar. 29, 2012.
(60) Provisional application No. 61/650,891, filed on May 23, 2012.

(51) Int. Cl. C07K 16/24; C07K 14/47; A61K 31/573; A61K 45/06; C12N 15/113; A61K 39/395; A61K 31/7088; C07K 16/38; A61K 38/17; A61K 38/21

(52) U.S. Cl. CPC C07K 16/245; A61K 38/1793; C07K 14/47; A61K 31/573; A61K 38/217; C12N 15/1136; A61K 39/3955; A61K 31/7088; C07K 16/24; C07K 16/38; A61K 45/06; A61K 2039/505
USPC 424/85.5; 514/19.8; 424/133.1; 424/142.1; 424/134.1; 514/171; 514/44 A; 424/94.6; 424/94.63; 514/19.5

(57) ABSTRACT

The invention includes a method of preventing or treating metastasis in a subject diagnosed with cancer, the method comprising determining whether at least one gene encoding one or more proteins is upregulated in a cancer tissue sample from the subject as compared to the level of expression of the at least one gene in a non-cancer control sample of the same tissue, and, if the at least one gene is upregulated in the cancer tissue sample from the subject, administering to the subject a therapeutically effective amount of a pharmaceutical composition comprising one or more protein depleting agents.

### CROSS-REFERENCE TO RELATED APPLICATIONS

The present application claims priority under 35 U.S.C. §119(e) to U.S. Provisional Applications No. 61/617,447, filed Mar. 29, 2012 and No. 61/650,891, filed May 23, 2012, all of which applications are hereby incorporated by reference in their entireties herein.

### BACKGROUND OF THE INVENTION

Metastasis or metastatic disease is the spread of a disease from one organ or part to another non-adjacent organ or part. Metastatic disease is primarily but not uniquely associated with malignant tumor cells and infections (Klein, 2008, Science 321(5897): 1785-88; Chiang & Massagué, 2008, New Engl. J. Med. 359(26):2814-23).

Cancer occurs after a single cell in a tissue is genetically damaged in ways that result in the formation of a putative cancer stem cell possessing a malignant phenotype. These cancer stem cells are able to undergo uncontrolled abnormal mitosis, which serves to increase the total number of cancer cells at that location. When the area of cancer cells at the originating site become clinically detectable, it is called primary tumor. Some cancer cells also acquire the ability to penetrate and infiltrate surrounding normal tissues in the local area, forming a new tumor. The newly formed tumor in the adjacent site within the tissue is called a local metastasis.

Some cancer cells acquire the ability to penetrate the walls of lymphatic and/or blood vessels, after which they are able to circulate through the bloodstream (circulating tumor cells) to other sites and tissues in the body. This process is known (respectively) as lymphatic or hematogenous spread. After the tumor cells come to rest at another site, they re-penetrate through the vessel or walls (extravasion), continue to multiply, and eventually another clinically detectable tumor is formed. This new tumor is known as a metastatic (or secondary) tumor. Metastasis is one of the hallmarks of malignancy. Most tumors and other neoplasms can metastasize, although in varying degrees (e.g., basal cell carcinoma rarely metastasizes) (Kumar et al., 2005, “Robbins and Cotran's Pathologic Basis of Disease”, 7th ed., Philadelphia: Elsevier Saunders).

Metastatic tumors are very common in the late stages of cancer. The most common places for the metastases to occur are the lungs, liver, brain, and the bones. There is also a propensity for certain tumors to seed in particular organs. For example, prostate cancer and breast cancer usually metastasizes to the bones. Colon cancer has a tendency to metastasize to the liver. Stomach cancer often metastasizes to the ovaries in women.

Studies have suggested that these tissue-selective metastasis processes are due to specific anatomic and mechanical routes. In particular, a cancer cell may colonize other organs and progress into macroscopic lesions only if its genotypic and phenotypic features are somehow compatible with the local microenvironment. Thus, the identification of the molecular determinants underpinning cancer cells colonization of secondary organs may reveal novel therapeutic targets to effectively counteract metastatic disease.

Interleukin-1 beta (IL-1β; SEQ ID NO:1), also known as catabolin, is a cytokine protein, which in humans is encoded by the IL1B gene (Auron et al., 1984, PNAS USA 81(24):7907-11; March et al., 1985, Nature 315(6021):641-7; Clark et al., 1986, Nucleic Acids Res. 14 (20):7897-914; Bensi et al., 1987, Gene 52(1):95-101). IL-1β is a member of the interleukin 1 cytokine family and produced by activated macrophages as a pro-protein, which is proteolytically processed to its active form by caspase 1 (CASP1/CE). This cytokine is an important mediator of the inflammatory response, and involved in cellular activities including cell proliferation, differentiation, and apoptosis. Studies associate the gene with susceptibility to schizophrenia (“Gene Overview of All Published Schizophrenia-Association Studies for IL1B”—SZgcne database, Schizophrenia Research Forum) and keratoconus (Kim et al., 2008, Mol. Vis. 14:2109-16).

IL-1β is a cytokine with pleiotropic effects and its production by the stroma has been previously correlated with local tumorigenesis and unfavorable prognosis (Apte et al., 2000, Adv. Exp. Med. Biol, 479:277-288). Constitutive expression of IL-1β in prostate cancer cells reduces expression of both PSA and AR (Abdul & Hoosein, 2000, Cancer Lett. 149:37-42).

CXCL6 (SEQ ID NO:3), which is encoded in humans by the CXCL6 gene, is a chemoattractant for neutrophils and has angiogenic properties, in contrast to most of CXC chemokines, which attract lymphocytes and exert an angiostatic effect (Stricter et al., 1995, Shock (Augusta, Ga.) 4:155-160).

Elafin (SEQ ID NO:4), which in humans is encoded by the PI3 gene, is an antiprotease with a major role in defending tissues from the damaging action of neutrophils' elastase (Williams et al., 2006, Clinical Science (London, England: 1979, Vol. 110, 21-35). Elafin has been previously found over-expressed in ovarian carcinoma upon inflammatory conditions and proposed to be a negative prognostic factor for this tumor (Yin et al., 2007, Mol. Cell. Biol. 27:7538-7550).

There is a need in the art to develop a novel method that identifies a subject that is at risk of or has developed metastatic cancer, and provides treatment that avoids, delays or minimizes the development of metastatic tumors in that subject, especially in the context of metastatic bone cancer associated with primary prostate or breast cancers. The present invention fulfills this need.

### BRIEF SUMMARY OF THE INVENTION

The invention includes a method of treating or preventing metastasis in a subject diagnosed with cancer. The method comprises determining whether at least one gene encoding a protein selected from the group consisting of IL-1β, CXCL6, Elafin and any combination thereof is upregulated in a cancer tissue sample from the subject as compared to the level of expression of the at least one gene in a non-cancer control sample of the same tissue, and, if the at least one gene is upregulated in the cancer tissue sample from the subject, administering to the subject a therapeutically effective amount of a pharmaceutical composition comprising a IL-1β-depleting agent and at least one therapeutic agent comprising a CXCL6-depleting agent or an Elafin-depleting agent, whereby the metastasis of the subject diagnosed with cancer is treated or prevented.

In one embodiment, the cancer comprises a solid cancer. In another embodiment, the solid cancer is selected from the group consisting of breast cancer and prostate cancer. In yet another embodiment, the metastasis comprises bone metastasis. In yet another embodiment, the at least one gene encodes IL-1β. In yet another embodiment, the at least one gene is upregulated by at least 50% as compared to the level of expression of the at least one gene in a non-cancer control sample of the same tissue.

In one embodiment, the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, an IL-1β antibody, siRNA, ribozyme, an antisense, an aptamer, a peptidomimetic, a small molecule, and a combination thereof. In another embodiment, the IL-1β antibody comprises an antibody selected from the group comprising a polyclonal antibody, monoclonal antibody, humanized antibody, synthetic antibody, heavy chain antibody, human antibody, biologically active fragment of an antibody, and any combination thereof.

In one embodiment, the CXCL6-depleting agent is a CXCL6 antibody selected from the group consisting of a polyclonal antibody, monoclonal antibody, humanized antibody, synthetic antibody, heavy chain antibody, human antibody, biologically active fragment of an antibody, and any combination thereof.

In one embodiment, the Elafin-depleting agent is an Elafin antibody selected from the group consisting of a polyclonal antibody, monoclonal antibody, humanized antibody, synthetic antibody, heavy chain antibody, human antibody, biologically active fragment of an antibody, and any combination thereof.

In one embodiment, the at least one therapeutic agent comprises a CXCL6-depleting agent. In another embodiment, the at least one therapeutic agent comprises a CXCL6-depleting agent and an Elafin-depleting agent.

In one embodiment, the method further comprises administering to the subject an additional compound selected from the group consisting of a chemotherapeutic agent, an anti-cell proliferation agent and any combination thereof. In another embodiment, the chemotherapeutic agent comprises an alkylating agent, nitrosourea, antimetabolite, antitumor antibiotic, plant alkyloid, taxane, hormonal agent, bleomycin, hydroxyurea, L-asparaginase, or procarbazine. In yet another embodiment, the anti-cell proliferation agent comprises granzyme, a Bcl-2 family member, cytochrome C, or a caspase. In yet another embodiment, the pharmaceutical composition and the additional compound are co-administered to the subject. In yet another embodiment, the pharmaceutical composition and the additional compound are co-formulated and co-administered to the subject. In yet another embodiment, the pharmaceutical composition is administered to the subject by an administration route selected from the group consisting of inhalational, oral, rectal, vaginal, parenteral, topical, transdermal, pulmonary, intranasal, buccal, ophthalmic, intrathecal, and any combinations thereof in yet another embodiment, the subject is a mammal. In yet another embodiment, mammal is a human.

The invention also comprises a kit comprising a IL-1β-depleting agent and at least one therapeutic agent comprising a CXCL6-depleting agent or an Elafin-depleting agent, an applicator, and an instructional material for use thereof.

In one embodiment, the instructional material comprises instructions for preventing or treating metastasis in a subject diagnosed with cancer, wherein the instructional material recites that the level of expression of at least one gene encoding a protein selected from the group consisting of IL-1β, CXCL6. Elafin and any combination thereof in a cancer tissue sample from the subject is compared to the levels of expression of the at least one gene in a non-cancer control sample of the same tissue. In another embodiment, the instructional material further recites that, if the at least one gene is upregulated in the cancer tissue sample from the subject, the subject is administered a therapeutically effective amount of a pharmaceutical composition comprising the IL-1β-depleting agent and the at least one therapeutic agent comprising a CXCL6-depleting agent or an Elafin-depleting agent.

In one embodiment, the cancer comprises ovarian cancer or prostate cancer. In another embodiment, the metastasis comprises bone metastasis. In yet another embodiment, the subject is a mammal. In yet another embodiment, the mammal is human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A-1D, illustrates the different bone-metastatic potential of the human prostate cancer cell lines used in the animal model (FIG. 1A) and the correlation between expression of PDGFRα and bone-metastatic potential (FIG. 1B). After being inoculated in the arterial circulation, PC3-ML and PC3-N(Rα) produce metastatic bone lesions in 90% and 75% of mice, respectively. PC3-N cells showed weak metastatic potential and generated bone lesions in only 20% of animals inspected three weeks post-inoculation. No lesions were detected in mice inoculated with PC3-N cells and inspected four weeks later, which suggest that the small number of lesions detected at three weeks eventually regress. DU-145 and DU-145(Rα) cells never progressed past the stage of Disseminated Tumor Cells (DTCs) and were detected only within three days post-inoculation (FIG. 1A). DU-145 as DTCs in the tibia of an inoculated animal (FIG. 1C); large metastatic tumors produced by PC3-ML cells in the femur (left) and spine (right) of a mouse inoculated four weeks earlier (FIG. 1D).

FIG. 2, comprising FIGS. 2A-2C, illustrates microarray data analysis for genes differentially expressed between PC3-ML and PC3-N cells (FIG. 2A) and between PC3-N and PC3-NRα (FIG. 2B). FIG. 2C, top: Venn diagram showing 7 genes that were found overlapping between the 16 and 40 genes identified above. FIG. 2C, bottom: the newly-identified genes directly correlate with high levels of PDGFRα expression and the bone-metastatic progression of PC3 cells.

FIG. 3, comprising FIGS. 3A-3C, illustrates microarray data analysis for differentially expressed genes between DU145Ra and DU45 cells (FIG. 3A) and the Venn diagram of the overlapping 7 genes associated with PDGFRα expression in the cell lines analyzed (FIGS. 3B-3C).

FIG. 4, comprising FIGS. 4A-4D, illustrates a bone-metastatic tumor produced by PC3-N cells exogenously expressing PDGFRβ (FIG. 4A) microarray data analysis for differentially expressed genes between PC3-NRβ and PC3-N cells (FIGS. 4B-4C) and a Venn diagram showing the overlapping genes between PC3-NRβ cells and the 7-genes set identified before (FIG. 4D).

FIG. 5, comprising FIGS. 5A-5C, illustrates microarray data analysis (FIG. 5A) for differentially expressed genes in PC3-ML clone 1 and PC3-N cells, PC3-ML clone 3 and PC3-N cells and a Venn diagram (FIG. 5B) showing the 3 overlapping genes found are associated with the bone-metastatic phenotype in all human prostate cancer cell lines used in the present model. FIG. 5C is a bar graph illustrating average tumor area observed when Clone 1 and Clone 3 were inoculated in five mice each and generated large skeletal lesions in all animals.

FIG. 6, comprising FIGS. 6A-6C, illustrates experiments in which IL-1β, one of the three putative bone-metastatic genes identified, was silenced by RNAi in PC3-ML cells (FIG. 6A) and induced a significant reduction in the area of bone metastatic lesions generated by these cells in SCID mice (FIGS. 6B-6C). In highly metastatic PC3-ML cells, RNA interference reduced both the protein expression of IL-1β precursor (FIG. 6A, top) and secretion of the mature form as measured by ELISA (FIG. 6A, bottom); four weeks after intracardiac inoculation of PC3-ML or PC3-ML(sh-IL-1β) cells all mice had developed bone metastatic tumors (FIG. 6B, top); however, the lesions generated by PC3-ML (sh-IL-1β) cells were significantly smaller in size (FIG. 6B, bottom, and FIG. 6C). PC3-ML cells transduced with a TRC lentiviral vector carrying a non-coding shRNA were used in control experiments in which 5 mice were euthanized 4 weeks post-inoculation and showed number and distribution of metastatic tumors comparable to parental PC3-ML cells (not shown). M=number of metastatic tumors. *** p=0.002

FIG. 7, comprising FIGS. 7A-7F, illustrates the effects of IL-1β over-expression on the metastatic potential of prostate cancer cells in vivo. IL-1β protein expression (FIG. 7A) and secretion (FIG. 7B, ELISA results) were exogenously increased in low-metastatic PC3-N cells; the resulting PC3-N(IL-1β) cells were as effective as PC3-N(Rα) cells in generating skeletal lesions in mice sacrificed four weeks after intracardiac inoculation (FIG. 7C), and produced bone lesions that were comparable in size (FIGS. 7D-7F). PC3-N cells transduced with an empty pLXSN vector were inoculated in the arterial circulation of five mice that were cuthanized four weeks later and found to be free of skeletal tumors (not shown). M=number of metastatic tumors.

FIG. 8, comprising FIGS. 8A-8C, illustrates experiments performed with DU145 cells, which similarly to PC3-N cells acquired bone-metastatic potential upon over-expression (FIG. 8A), generating skeletal metastases in 40% of inoculated SCID mice (FIG. 8C). The expression (FIG. 8A) and secretion (by ELISA, FIG. 8B, top) of IL-1β were exogenously induced in DU-145 cells, which are normally negative for this protein and non-metastatic; the resulting DU-145 (IL-1β) cells generated bone lesions in 40% of mice inoculated via the intracardiac route and sacrificed either 2 or 4 weeks post-inoculation (FIG. 8B, middle); the size of skeletal lesions increased in a time-dependent manner, thus suggesting metastatic progression (FIG. 8B, bottom). M=number of metastatic tumors. *p=0.037; size comparison of bone tumors detected at 2 weeks post-inoculation in mice that received PC3-N(IL-1β) or DU-145 (IL-1β) cells (FIG. 8C).

FIG. 9, comprising FIGS. 9A-9E, illustrates upregulation of the genes for IL-1β, CXCL6 and Elafin in prostate cancer and correlation of IL-1β protein expression with Gleason scores. The Oncomine database showed a consistent increase in the expression of these three genes in tumor as compared to normal prostate tissue (FIG. 9A); a significant correlation between IL-1β and CXCL6 expression in tumors with Gleason scores (7-9) and (8-9), respectively. Only one study analyzing Elafin expression in tumor versus normal and Gleason scores (8-9) was available (FIG. 9B); TMAs including 227 cases of primary prostate adenocarcinoma were stained for IL-1β with signal intensities that were scored as weak (1+, top), moderate (2+, middle) and strong (3+, bottom) (FIG. 9C); higher magnification of two representative tissue specimens that stained negative (left) and strongly positive (right) for IL-1β, respectively. Hematoxylin-Eosin counterstaining was used (FIG. 9D); contingency table of TMA data showing that prostate tumors with Gleason scores (≧7) expressed higher levels of the IL-1β protein as compared to tumors with lower Gleason scores (<7) (FIG. 9E). Chi-square=33.08 and p<0.0001.

FIG. 10, comprising FIGS. 10A-10B, illustrates detection of IL-1β protein in skeletal metastases and correlation with PSA and synaptophysin expression. Seven specimens collected from different prostate cancer patients were analyzed. All specimens stained positive for IL-1β and the intensity of the signal appeared to be inversely correlated with the expression levels of PSA in the same areas. Representative images from two different tumor regions in a single patient are shown in FIG. 10A. Two out of seven specimens stained positive for both IL-1 and synaptophysin (FIG. 10B). Magnification: 100× for FIG. 10A; 200× for FIG. 10B.

FIG. 11 is a representation of a gel illustrating the over-expression of COX-2 induced by IL-1β secreted by bone-metastatic cancer cells. Human bone Mesenchymal Stem Cells (MSCs) were exposed for 48 hours to a medium conditioned by PC3-ML cells and showed an evident increase in COX-2 expression, which was inhibited by pre-treatment with the IL-1R inhibitor Anakinra (10 μg/ml). This effect was similar to that observed when MSCs were exposed directly to IL-1β (0.1 ng/ml).

FIG. 12, comprising FIGS. 12A-12C, illustrates the analysis of IL-1β expression and secretion from prostate cancer cells. PC3-ML cells showed higher levels of IL-1β expression as compared to PC3-N cells and the exogenous over-expression of PDGFRα up-regulated IL-1β expression in PC3-N cells. In contrast, PDGFRα did not increase IL-1β expression in DU-145 cells (FIG. 12A); the levels of IL-1β secreted by different cell types and measured by ELISA confirmed the Western blotting results. All together these data provide full validation of the gene expression microarray analyses (FIG. 12B); over-expression or silencing of IL-1β in different cell types that were tested in the animal model for their respective metastatic behavior (FIG. 12C).

FIG. 13, comprising FIGS. 13A-13B, illustrates the finding that PDGFRα regulates Elafin and CXCL6 expression in prostate cancer cells, as demonstrated by ELISA. PC3-ML cells express higher levels of Elafin as compared to PC3-N cells and the over-expression of PDGFRα upregulates this protein in PC3-N cells but not in DU-145 cells (FIG. 13A); a similar pattern of PDGFRα regulation can be observed for CXCL6 when measured as secreted protein in the same cells (FIG. 13B). Both sets of data were in full agreement with the results of the microarray analysis.

FIG. 14, comprising FIGS. 14A-14B, illustrates the results of in vivo experiments conducted using human prostate cancer cells double-KO for IL-1β and CXCL6.

### DETAILED DESCRIPTION OF THE INVENTION

The present invention relates to the unexpected discovery that prostate cancer cells metastasizing to the skeleton via the hematogenous route upregulate the genes coding for the cytokine interleukin-1beta (IL-1β), the chemokine CXCL6 or GCP-2, and the protease inhibitor Elafin (PI3). This three-gene set is regulated by the alpha-receptor for Platelet-Derived Growth Factor (PDGFRα).

The studies reported herein indicated that the exogenous over-expression of IL-1β alone induced the progression of non-metastatic disseminated tumor cells (DTC) into full-blown skeletal lesions, whereas IL-1β knockdown significantly impaired bone-metastatic DTCs. IL-1β secreted by metastatic cells induced the over-expression of COX-2 in human bone mesenchymal cells treated with conditioned media from bone metastatic prostate cancer cells. Inspection of human tissue specimens from skeletal metastases detected prostate cancer cells positive for both IL-1β and synaptophysin while concurrently lacking prostate specific antigen (PSA) expression. IL-1β expression directly correlated to high Gleason grades (e.g., Gleason scores≧7) and metastasis. The studies further showed that bone-metastatic cancer cells secreted IL-1β and supported the intra-osseous survival and progression of non-metastatic phenotypes. In other words, IL-1β supports the skeletal colonization and metastatic progression of prostate cancer cells with an acquired neuroendocrine phenotype.

These findings collectively indicated that IL-1β, either by itself or in concert with IL-1β-regulated CXCL6 and Elafin, support the progression of prostate cancer skeletal metastases. In one embodiment, upregulation of the gene encoding IL-1β correlates with metastasis. In another embodiment, upregulation of the genes encoding IL-1β and CXCL6 correlates with metastasis. In yet another embodiment, upregulation of the genes encoding IL-1β and Elafin correlates with metastasis. In yet another embodiment, upregulation of the genes encoding CXCL6 and Elafin correlates with metastasis. In yet another embodiment, upregulation of the genes encoding IL-1β, CXCL6 and Elafin correlates with metastasis.

The invention includes a method of preventing or treating metastasis in a subject diagnosed with cancer. According to the method, one determines whether at least one gene encoding a protein selected from the group consisting of IL-1β, CXCL6, Elafin and any combination thereof are upregulated in a cancer tissue sample from the subject as compared to the level of expression of the at least one gene in a non-cancer control sample of the same tissue. If the at least one gene is upregulated in the cancer tissue sample, a therapeutically effective amount of a pharmaceutical composition comprising a IL-1β-depleting agent and at least one therapeutic agent comprising a CXCL6-depleting agent or a Elafin-depleting agent is administered to the subject, whereby IL-1β, CXCL6 and/or Elafin are depleted in the subject and whereby metastasis is prevented or treated in the subject. In one embodiment, the cancer comprises a solid cancer. In another embodiment, the solid cancer is selected from the group consisting of breast cancer and prostate cancer. In yet another embodiment, the metastasis comprises bone metastasis. In yet another embodiment, the at least one gene is upregulated by at least 50% as compared to the level of expression of the at least one gene in a non-cancer control sample of the same tissue. In yet another embodiment, the at least one gene is upregulated by at least 100% as compared to the level of expression of the at least one gene in a non-cancer control sample of the same tissue.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element.

A “subject” or “individual” or “patient,” as used therein, can be a human or non-human mammal. Non-human mammals include, for example, livestock and pets, such as ovine, bovine, porcine, canine, feline and murine mammals. Preferably, the subject is human.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “DTC” refers to a disseminated tumor cell.

As used herein, the term “MSC” refers to a mesenchymal stem cell.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “non-cancer control sample” as relating to a subject's tissue refers to a sample from the same tissue type, obtained from the patient, wherein the sample is known or found not to be afflicted with cancer. For example, a non-cancer control sample for a subject's lung tissue refers to a lung tissue sample obtained from the subject, wherein the sample is known or found not to be afflicted with cancer. “Non-cancer control sample” for a subject's tissue also refers to a reference sample from the same tissue type, obtained from another subject, wherein the sample is known or found not to be afflicted with cancer. “Non-cancer control sample” for a subject's tissue also refers to a standardized set of data (such as, but not limited to, identity and levels of gene expression, protein levels, pathways activated or deactivated etc), originally obtained from a sample of the same tissue type and thought or considered to be a representative depiction of the non-cancer status of that tissue.

As used herein, the term “treatment” or “treating” is defined as the application or administration of a therapeutic agent. i.e., a compound useful within the invention (alone or in combination with another pharmaceutical agent), to a subject, or application or administration of a therapeutic agent to an isolated tissue or cell line from a subject (e.g., for diagnosis or ex vivo applications), who has cancer, a symptom of cancer or the potential to develop cancer, with the purpose to cure, heal, alleviate, relieve, alter, remedy, ameliorate, improve or affect cancer, the symptoms of cancer or the potential to develop cancer. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. Disease and disorder are used interchangeably herein.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the term “IL-1β-depleting agent” refers to an agent that reduces the expression, production and/or circulating concentration of IL-1β in a subject treated with such agent. In one embodiment, the agent is an antibody that binds to and neutralizes IL-1β. In another embodiment, the agent is a chemical compound that inhibits formation of IL-1β. In yet another embodiment, the agent reduces the expression or production of IL-1β in the subject. Agents that reduce the expression, production and/or circulating concentration of IL-1β by any physiological mechanism are considered useful within the methods of the invention. In one embodiment, the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, and any combinations thereof.

As used herein, the term “CXCL6-depleting agent” refers to an agent that reduces the expression, production and/or circulating concentration of CXCL6 in a subject treated with such agent. In one embodiment, the agent is an antibody that binds to and neutralizes CXCL6. In another embodiment, the agent is a chemical compound that inhibits formation of CXCL6. In yet another embodiment, the agent reduces the expression or production of CXCL6 in the subject. Agents that reduce the expression, production and/or circulating concentration of CXCL6 by any physiological mechanism are considered useful within the methods of the invention.

As used herein, the term “Elafin-depleting agent” refers to an agent that reduces the expression, production and/or circulating concentration of Elafin in a subject treated with such agent. In one embodiment, the agent is an antibody that binds to and neutralizes Elafin. In another embodiment, the agent is a chemical compound that inhibits formation of Elafin. In yet another embodiment, the agent reduces the expression or production of Elafin in the subject. Agents that reduce the expression, production and/or circulating concentration of Elafin by any physiological mechanism are considered useful within the methods of the invention.

As used herein, the terms “effective amount” or “therapeutically effective amount” or “pharmaceutically effective amount” of a compound are used interchangeably to refer to the amount of the compound that is sufficient to provide a beneficial effect to the subject to which the compound is administered. The term to “treat.” as used herein, means reducing the frequency with which symptoms are experienced by a patient or subject or administering an agent or compound to reduce the severity with which symptoms are experienced. An appropriate therapeutic amount in any individual case may be determined by one of ordinary skill in the art using routine experimentation. By the term “specifically bind” or “specifically binds,” as used herein, is meant that a first molecule (e.g., an antibody) preferentially binds to a second molecule (e.g., a particular antigenic epitope), but does not necessarily bind only to that second molecule.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; ester, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener, hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

The term “antibody,” as used herein, refers to an immunoglobulin molecule able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

By the term “synthetic antibody” as used herein, is meant an antibody generated using recombinant DNA technology, such as, for example, an antibody expressed by a bacteriophage as described herein. The term should also be construed to mean an antibody generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

By the term “applicator,” as the term is used herein, is meant any device including, but not limited to, a hypodermic syringe, a pipette, and the like, for administering the compounds and compositions of the invention.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopts highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

As used herein “endogenous” refers to any material from or produced inside an organism, cell, tissue or system.

As used herein, the term “exogenous” refers to any material introduced from or produced outside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer. The term “protein” typically refers to large polypeptides. The term “peptide” typically refers to short polypeptides. Conventional notation is used herein to portray polypeptide sequences: the left-hand end of a polypeptide sequence is the amino-terminus; the right-hand end of a polypeptide sequence is the carboxyl-terminus. As used herein, a “peptidomimetic” is a compound containing non-peptidic structural elements that is capable of mimicking the biological action of a parent peptide. A peptidomimetic may or may not comprise peptide bonds.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc. as well as individual numbers within that range, for example, 1, 2, 2.7, 3, 4, 5, 5.3, and 6. This applies regardless of the breadth of the range.

### Compositions

In one embodiment, a composition useful within the methods of the invention comprises at least one therapeutic agent selected from the group consisting of a IL-1β-depleting agent, a CXCL6-depleting agent, an Elafin-depleting agent and any combinations thereof. In another embodiment, a composition useful within the methods of the invention comprises an IL-1β-depleting agent and at least one therapeutic agent comprising a CXCL6-depleting agent or an Elafin-depleting agent. In one embodiment, the at least one therapeutic agent comprises a CXCL6-depleting agent. In another embodiment, the at least one therapeutic agent comprises an Elafin-depleting agent. In yet another embodiment, the at least one therapeutic agent comprises a CXCL6-depleting agent and an Elafin-depleting agent.

The agent contemplated within the invention reduces the expression, production and/or circulating concentration of IL-1β, CXCL6 and/or Elafin in a subject. In one embodiment, the agent is an antibody that binds to and neutralizes IL-1β, CXCL6 and/or Elafin. In another embodiment, the agent is a chemical compound that inhibits formation of IL-1β, CXCL6 and/or Elafin. In yet another embodiment, the agent reduces the expression or production of IL-1β, CXCL6 and/or Elafin in the subject. In yet another embodiment, the agent is selected from the group consisting of anakinra, XOMA-052, AMG-108, canakinumab, rilonacept, K-832, CYT-013-IL1bQb, Y-2189102, dexamethasone, interferon-gamma, pentoxifylline, and any combinations thereof. Agents that reduce the expression, production and/or circulating concentration of IL-1β, CXCL6 and/or Elafin by any physiological mechanism are considered useful within the methods of the invention.

Non-limiting examples of IL-1β-depleting agents contemplated within the methods of the invention are:

Dexamethasone (8S,9R,10S,11S,13S,14S,16R,17R)-9-Fluoro-11,17-dihydroxy-17-(2-hydroxyacetyl)-10,13,16-trimethyl-6,7,8,9,10,11,12,13,14,15,16,17-dodecahydro-3H-cycopenta[a]phenanthren-3-one) or a salt thereof: a known inhibitor of IL-1β production (Kern et al. 1998, J. Clin. Invest. 81:237-244);

Canakinumab (also known as Ilaris®, Novartis; previously ACZ885; Dhimolea, 2010, Mabs 2(1):3-13): a human monoclonal antibody targeted at interleukin-1 beta. It has no cross-reactivity with other members of the interleukin-1 family, including interleukin-1 alpha (Lachmann et al., 2009, New Engl. J. Med. 360(23):2416-25). Canakinumab was approved for the treatment of cryopyrin-associated periodic syndromes (CAPS) by the FDA on June 2009 and by the European Medicines Agency in October 2009. Canakinumab is also in clinical trials as a possible treatment for chronic obstructive pulmonary disease (Yasothan & Kar, 2008, Nat. Rev. Drug Discov. 7(4):285).

Rilonacept (also known as IL-1 Trap or Arcalyst®, Regeneron): an interleukin 1 inhibitor (Drug News Perspect. 21(4): 232). Rilonacept is a dimeric fusion protein consisting of the extracellular domain of human interleukin-1 receptor and the FC domain of human IgG1 that binds and neutralizes IL-1. Rilonacept is used for the treatment of cryopyrin-associated periodic syndromes (CAPS).

AMG-108: a fully human monoclonal antibody that targets inhibition of the action of interleukin-1 (Cardiel et al., Arthritis Res. Ther. 12(5):R192).

Anakinra (also known as Kineret®, Amgen): Anakinra is an IL-1 receptor antagonist (So et al., 2007, Arthritis Res. Ther. 9(2):R28). Anakinra is a recombinant, non-glycosylated version of human IL-1 receptor antagonist prepared from cultures of genetically modified E. coli. Anakinra blocks the biologic activity of naturally occurring IL-1, by competitively inhibiting the binding of IL-1 to the Interleukin-1 type receptor.

Interferon-gamma: known to selectively inhibit IL-1β gene expression (Chujor et al., 1996, Eur. J. Immun. 26:1253-1259).

Pentoxifylline: a known inhibitor of the synthesis of IL1-β (Roy et al., 2007, J. Toxicol. Environ. Health B Crit. Rev. 10(4):235-57; Zargari, 2008, Dermat. Online J. 14(11):2).

XOMA-052 (also known as gevukizumab): a potent anti-IL-1β humanized neutralizing antibody (Owyang et al., 2011, mAbs 3(1):49-60; U.S. Pat. No. 7,531,116 to Masat et al.). XOMA-052 has a 300 femtomolar binding affinity for human IL-1β and an in vitro potency in the low picomolar range. XOMA-052 has been shown to be active in mouse models of acute gout.

K-832 (also known as 2-benzyl-5-(4-chlorophenyl)-6-[4-(methylthio)phenyl]-2H-pyridazin-3-one): this compound has high inhibitory activity against production of interleukin-1β (i.e., acts as a IL-1β secretion inhibitor), and is being tested as a preventive and therapeutic drug for immune, inflammatory, and ischemic diseases (U.S. Pat. No. 6,348,468 to Ohkuchi et al.; Tabunoki et al., 2003, Arthritis Rheum. 48 (Suppl. S555); Miura et al., 2010, Eur. J. Pharm. Biopharm. 76(2):215-221).

CYT-013-IL1bQb (Cytos Biotechnology AG): a IL-1β vaccine (ClinicalTrials.gov., NCT00924105; http://www dot clinicaltrials dot gov/ct2/show/NCT00924105).

LY-2189102 (Eli Lilly): a humanized IgG4 monoclonal anti-IL1β antibody in development for the treatment of diabetes, with a binding affinity of 2.8 pM, and a half-life and bioavailability of 20.3 days and 55%, respectively, after SC administration to healthy volunteers. LY2189102 was recently studied in a Phase II study in T2DM patients (CT registry NCT00942188; ClinicalTrials.gov., 2011, “A safety and pharmacokinetics study in patients with rheumatoid arthritis”, at: http://www dot clinicaltrials dot gov/ct2/show/NCT00380744; ClinicalTrials dot gov., 2011, “A study for patients with rheumatoid arthritis on methotrexate (MTX) with an inadequate response to TNF inhibitor therapy”, at: http://www dot clinicaltrials dot gov/ct2/show/NCT00689728.

Further non-limiting examples of IL-1β-depleting agents contemplated within the methods of the invention are any IL-1β antibody, siRNA, ribozyme, antisense, aptamer, peptidomimetic, small molecule, and a combination thereof.

Further non-limiting examples of CXCL6-depleting agents contemplated within the methods of the invention are any CXCL6 antibody, siRNA, ribozyme, antisense, aptamer, peptidomimetic, small molecule, and a combination thereof.

Further non-limiting examples of Elafin-depleting agents contemplated within the methods of the invention are any Elafin antibody, siRNA, ribozyme, antisense, aptamer, peptidomimetic, small molecule, and a combination thereof.

### Description

The present invention relates to the discovery that prostate cancer cells metastasizing to the skeleton via the hematogenous route upregulate the genes encoding IL-1β, CXCL6 (GCP-2), and Elafin (PI3). This three-gene set is regulated by the alpha-receptor for Platelet-Derived Growth Factor (PDGFRα), implicated in the acquisition of bone-metastatic potential by prostate malignant phenotypes (Dolloff et al., 2005, Oncogene 24:6848-6854; Russell et al., 2009, Oncogene 28:412-421; Russell et al., 2010, Cancer Res. 70:4195-4203; Russell et al., 2010, Clin. Cancer Res. 16:5002-5010).

As described herein, the present experiments indicated that the exogenous over-expression of IL-1β alone induced the progression of non-metastatic DTC into full-blown skeletal lesions, whereas IL-1β knockdown significantly impaired bone-metastatic DTCs. Further, human tissue specimens showed that IL-1β expression was directly correlated to high Gleason grades (e.g., Gleason scores≧0.7) and metastasis. Bone-metastatic cancer cells were found to secrete IL-1β and support the intraosseus survival and progression of non-metastatic phenotypes. The co-expression of IL-1β with the NEPC marker synaptophysin in prostate cells detected in human skeletal lesions corroborates the idea that this cytokine plays a role in the progression of bone metastatic tumors affecting prostate cancer patients treated with androgen-deprivation therapy. The findings described herein collectively indicated that IL-1β, either by itself or in concert with IL-1β-regulated CXCL6 and Elafin, support, the progression of prostate cancer skeletal metastases.

In order to isolate the molecular factors determining the pro-metastatic role of PDGFRα, genome-wide expression analysis and comparative profiling were performed using malignant phenotypes that differ for the constitutive or over-expressed PDGFRα levels, as well as their bone-metastatic propensity in the animal model. First, genes differentially regulated in PC3-ML and PC3-N cells (Wang & Stearns, 1991, Differentiation: Res. Biol. Div. 48:115-125 (1991), two prostate cancer sub-lines derived from the PC3 parental line, were examined.

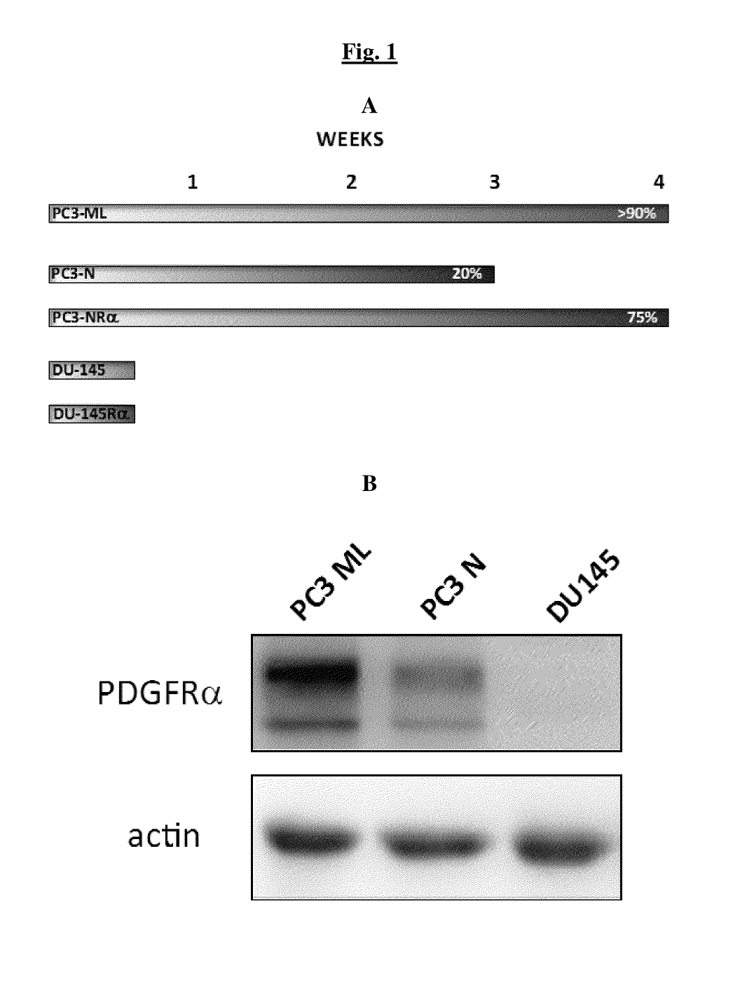

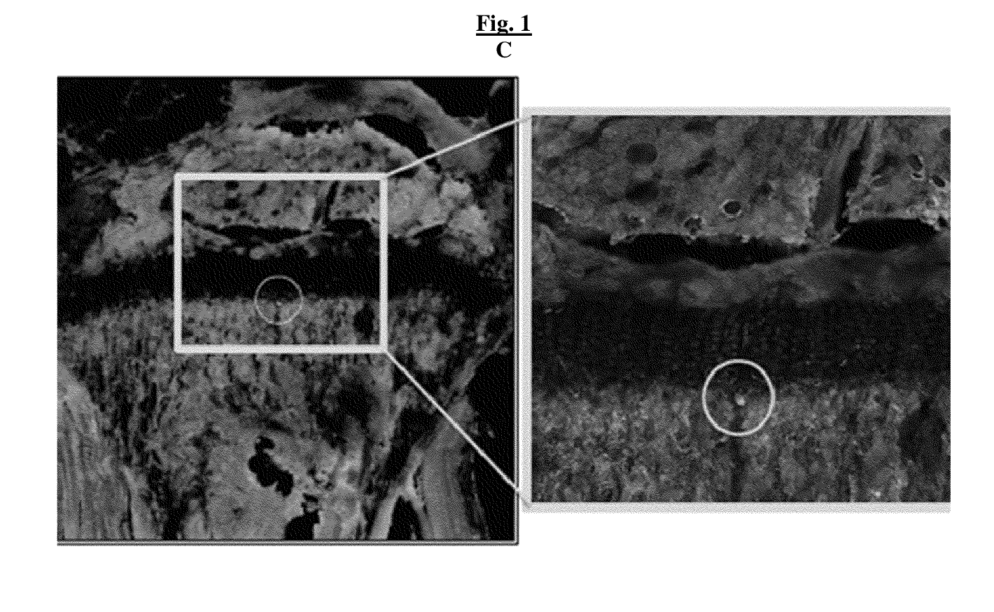

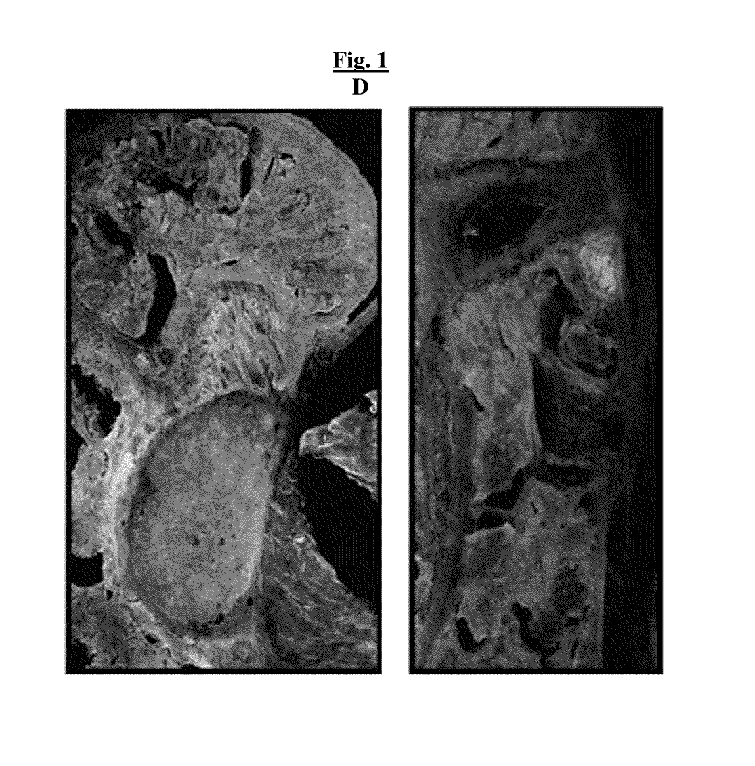

PC3 cells were originally isolated from a bone lesion in a patient with grade IV metastatic prostate adenocarcinoma (Kaighn et al., 1979, Invest. Urol. 17:16-23) and lack prostate specific antigen (PSA) and androgen receptor (AR) expression, both features observed in 20% and 40% of skeletal lesions from prostate cancer, respectively (Cheville et al., 2002, Cancer 95:1028-1036). The androgen-independent state of PC3 cells is associated with a neuroendocrine phenotype, which is frequently induced by the androgen withdrawal in prostate cancer patients (Hansson & Abrahamsson, 2001. Ann. Oncol. 12:145-152: Debes & Tindall, 2002, Cancer Lett. 187:1-7: Yang et al., 2009, Cancer Res. 69:151-160). The direct inoculation of PC3-ML cells in the arterial circulation of SCID mice generates skeletal lesions in more than 90% of animals (Russell et al., 2009, Oncogene 28:412-421) at four weeks post-inoculation (FIGS. 1A & 1D). These cells express high levels of PDGFRα, in contrast to PC3-N cells and DU-145 cells, which fail to metastasize via hematogenous route in the same animal model and express either significantly lower levels of the receptor or fail to express it, respectively (FIG. 1B).

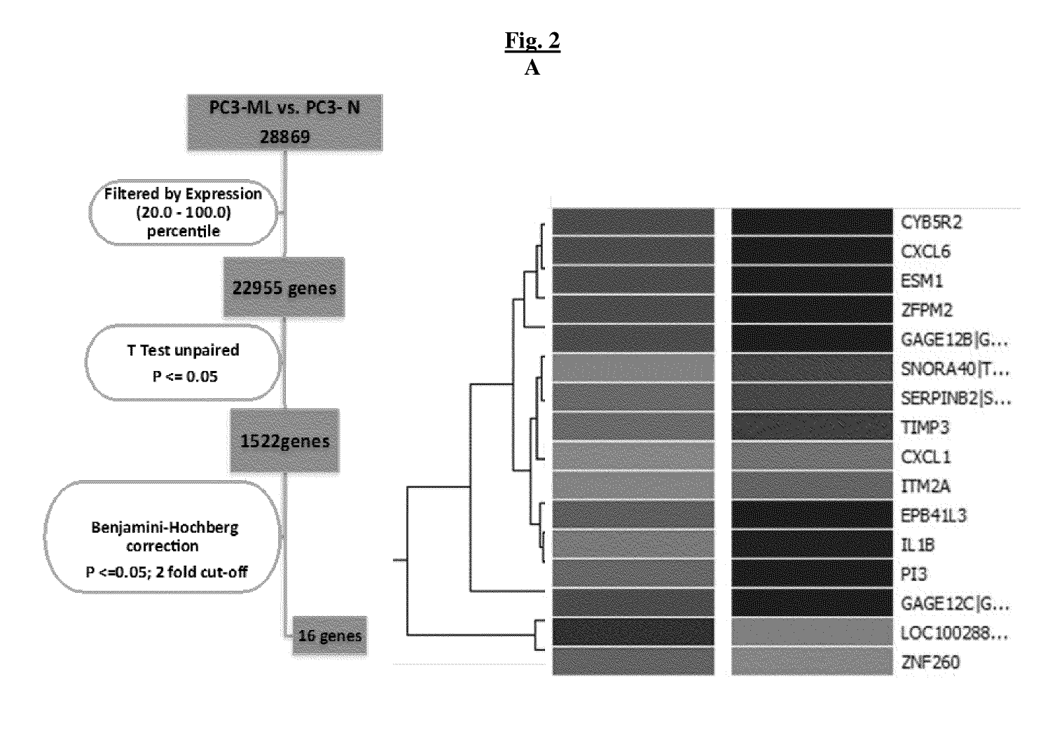

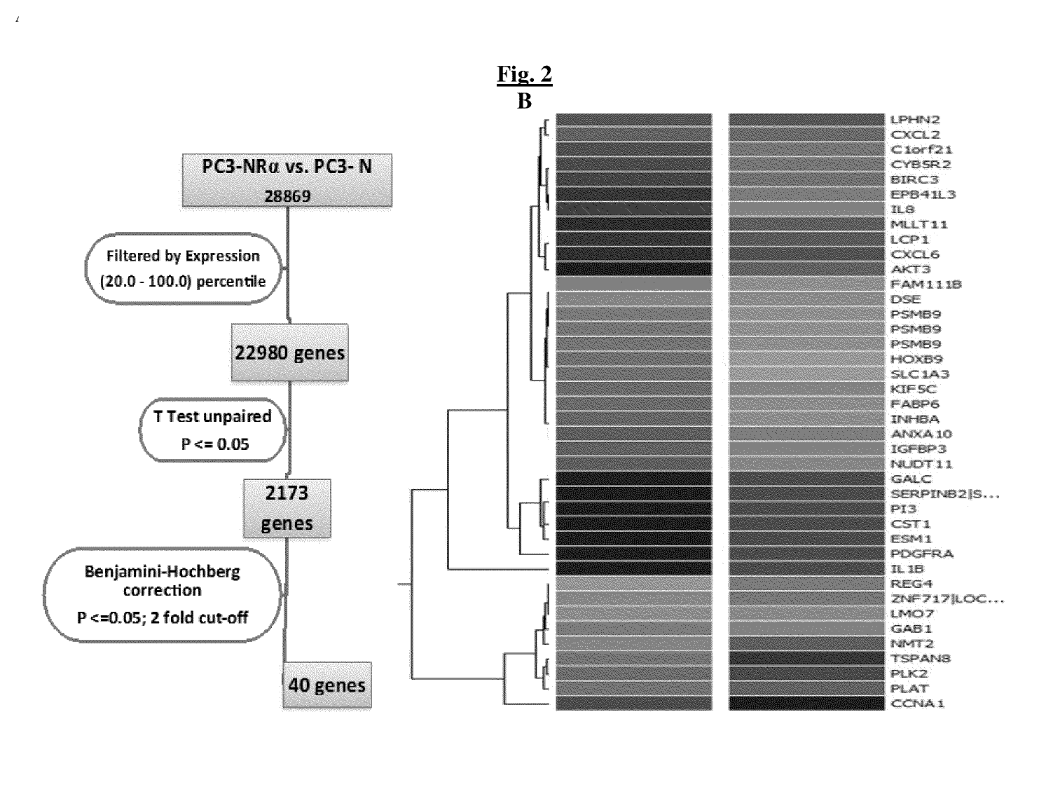

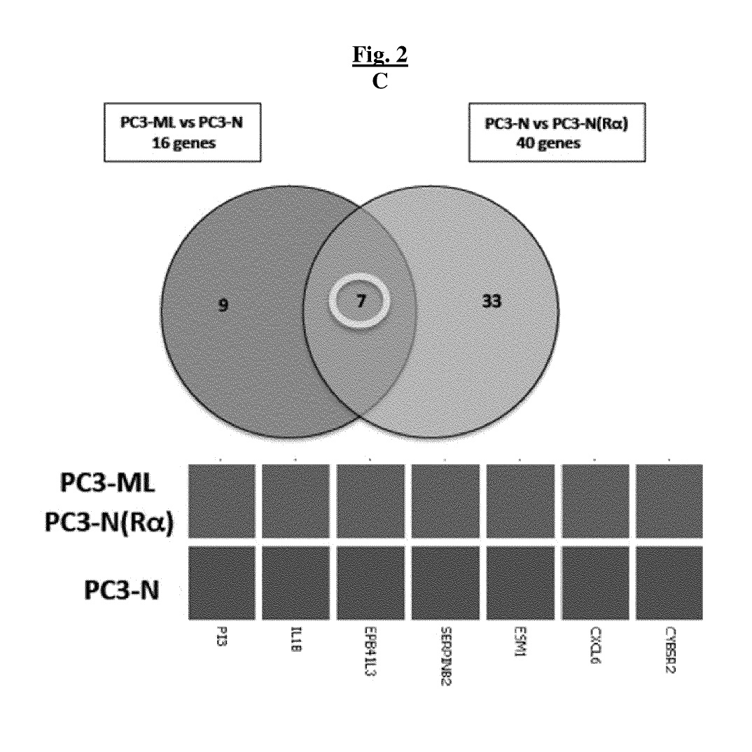

Microarrays data analysis revealed that 16 genes were differentially expressed between metastatic PC3-ML and non-metastatic PC3-N cells (FIG. 2A). To ascertain how many of these genes were regulated by PDGFRα expression and correlated with an increased bone-metastatic potential, the gene-expression profiles of PC3-N cells with PC3-N cells over-expressing exogenous PDGFRα (PC3-NRα) were compared, since PC3-NRα cells acquire a bone-metastatic phenotype indistinguishable from that of their PC3-ML counterpart when tested in the present animal model (FIG. 1A).

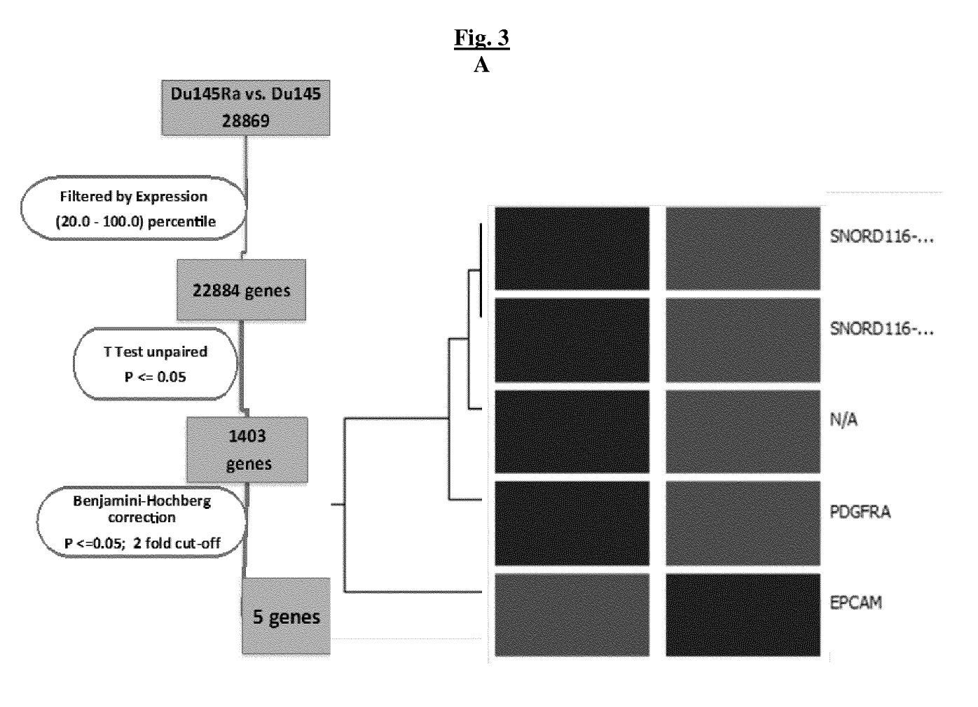

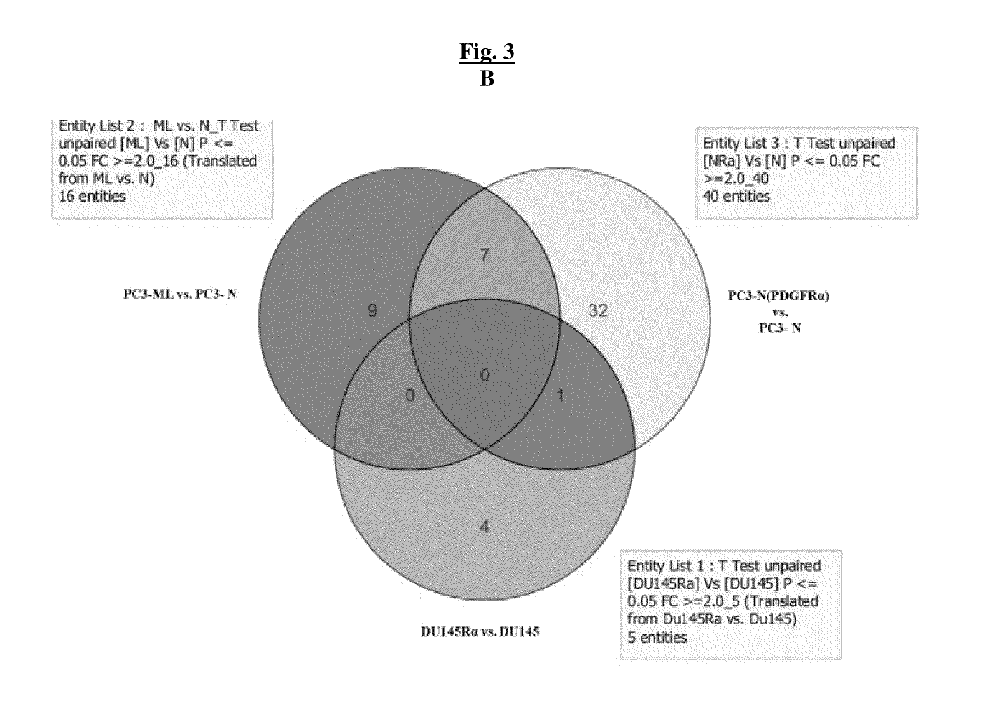

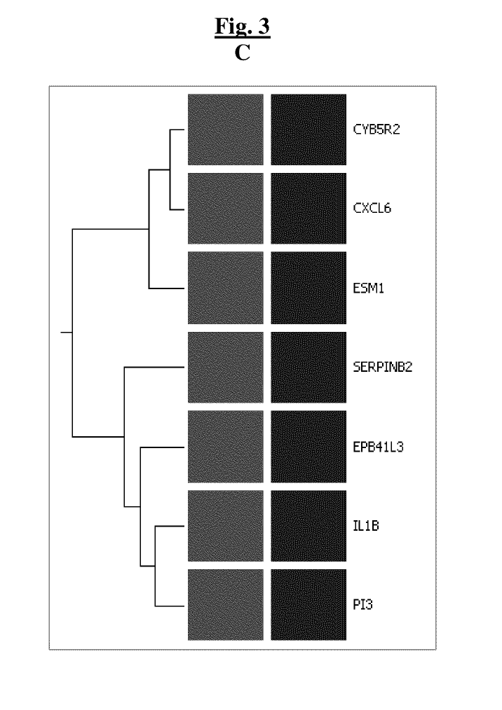

Forty genes that were differentially regulated by the over-expression of PDGFRα were identified in PC3-N cells (FIG. 2A), but only 7 were found overlapping with the 16 genes differentially regulated in PC3-ML and PC3-N cells (FIGS. 3B-3C). Based on the in vivo results, this observation suggested an underlying role for these 7 genes in the induction of metastatic potential by PDGFRα. Interestingly, DU-145 cells, originally derived from a prostate cancer brain metastasis lack endogenous PDGFRα expression (FIG. 1B) and fail to survive more than three days as DTCs in the bone marrow of the present mouse model (Russell et al., 2009, Oncogene 28:412-421). The exogenous expression of PDGFRα in DU-145 cells failed to induce a bone-metastatic phenotype and, in agreement with this observation, none of the 5 genes regulated by this receptor (FIG. 3A) overlapped the 16-gene or 7-gene sets identified as described above (FIGS. 3B-3C).

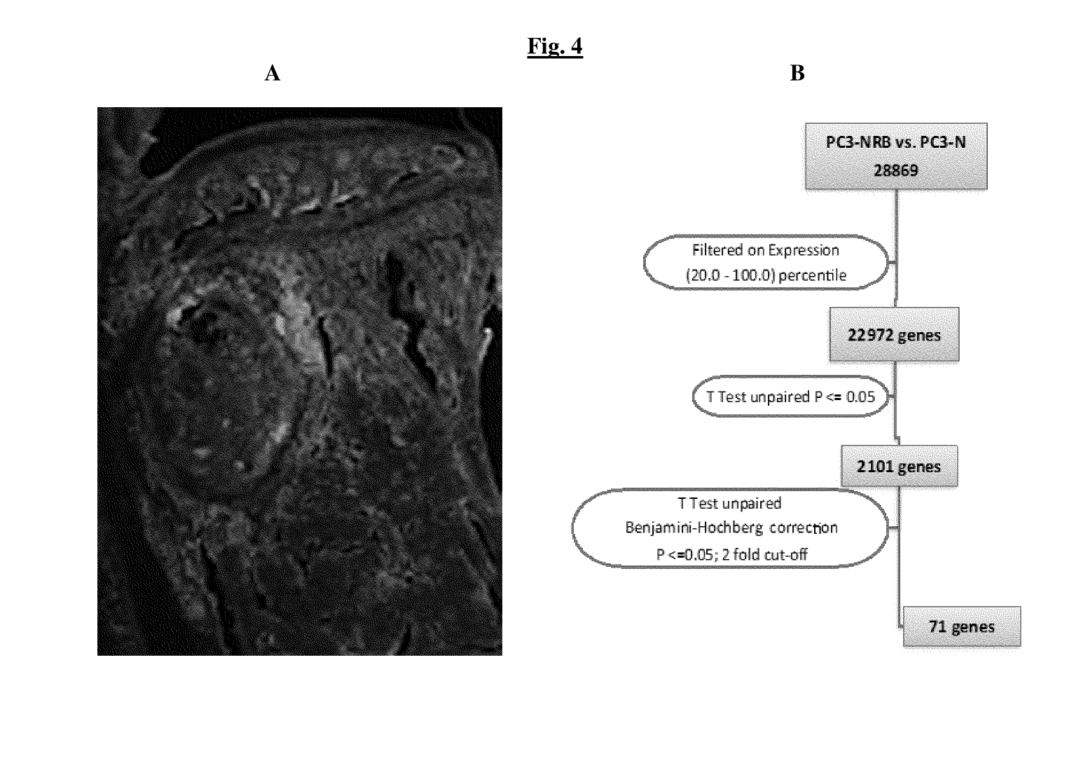

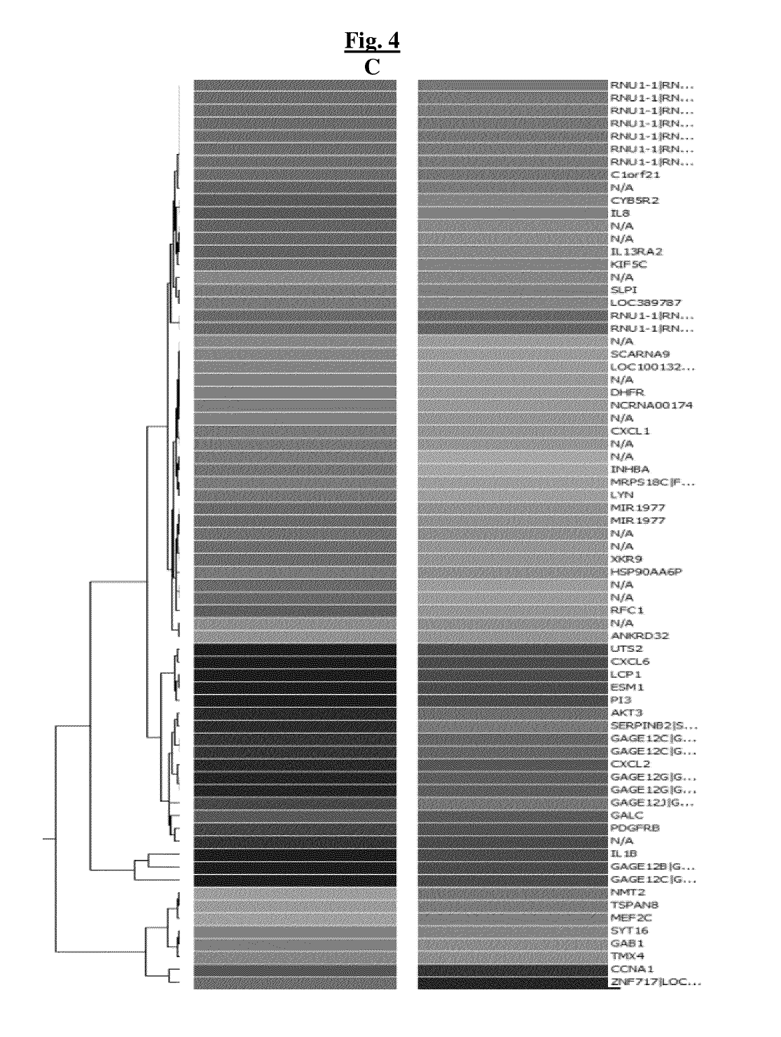

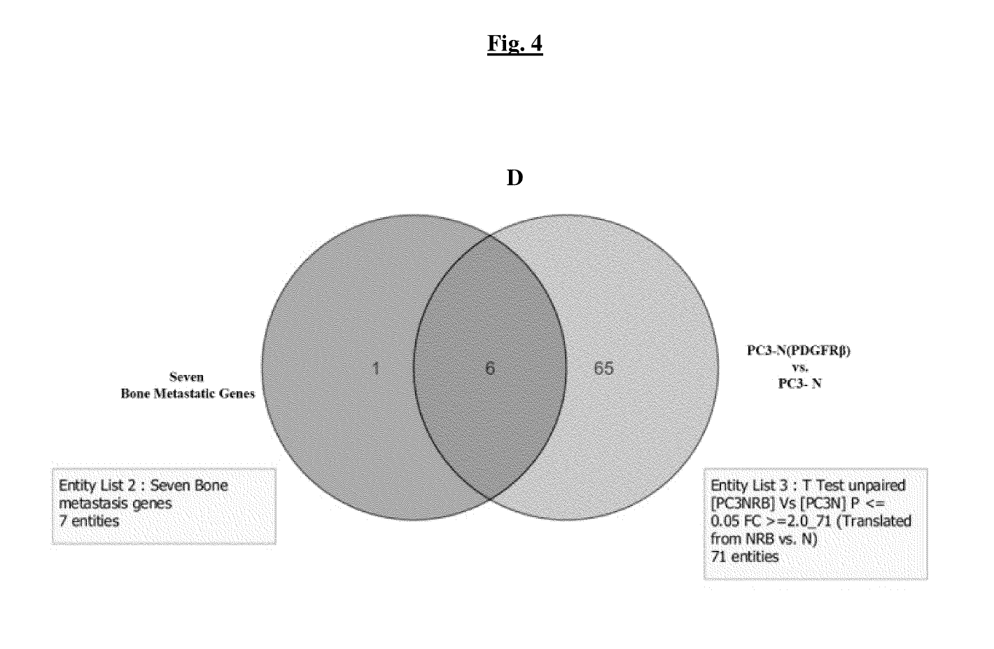

The next question addressed was whether the beta isoform of PDGFR (PDGFRβ) would exert the same effects on gene-expression profiles and metastatic potential as PDGFRα, given the high degree of structural homology between these two receptors (Claesson-Welsh et al., 1989. J. Biol. Chem. 264:1742-1747). PC3-N cells lack endogenous PDGFRβ (Dolloff et al., 2005, Oncogene 24:6848-6854), and its exogenous expression also promoted metastatic progression, as shown by the macroscopic lesions generated by PC3-NRβ cells disseminated to the bone of mice via the arterial circulation (FIG. 4A). Interestingly, the exogenous expression of PDGFRβ in PC3-N cells differentially regulated 71 (FIGS. 4B-4C) genes, of which 6 overlapped with the 7-gene set regulated by the α-isoform in the same cells (FIG. 4D). Taken together, these results not only implicate both PDGFRα and PDGFRβ in the progression of prostate DTCs in the skeleton, but also indicate that the acquisition of the bone-metastatic phenotype could be attributed to molecular interactions and recruitment of signaling pathways commonly shared by these two receptors.

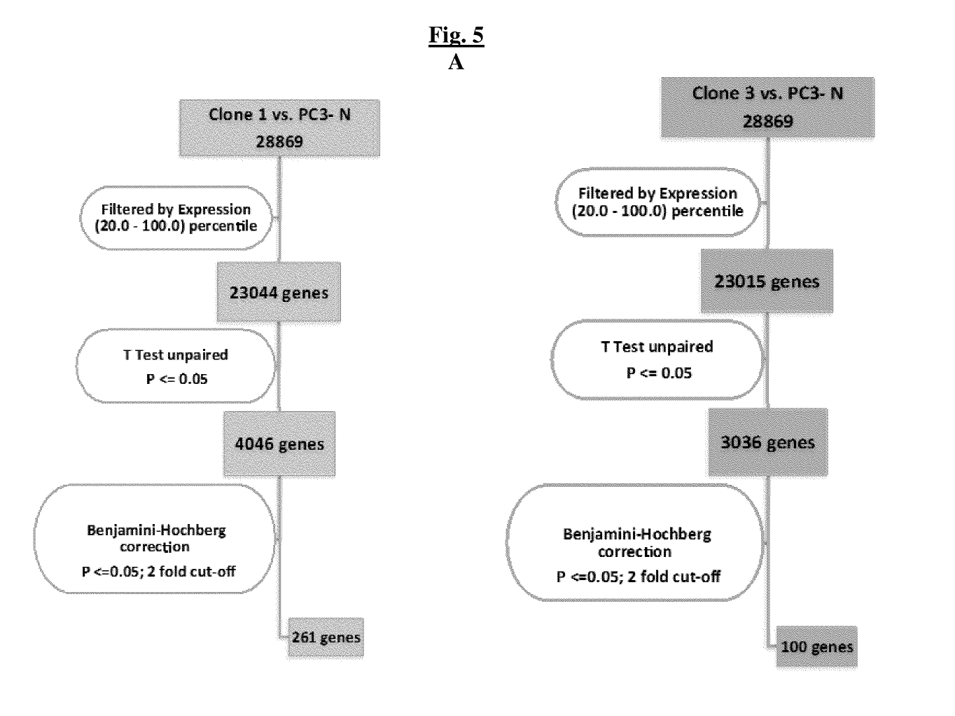

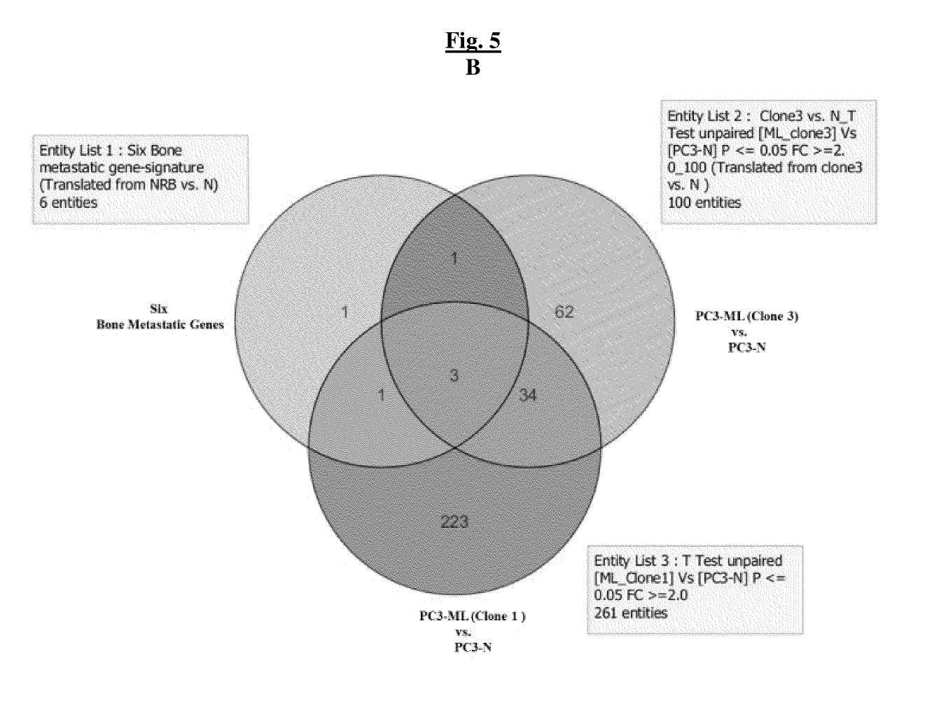

In order to segregate the most restricted cohort of pro-metastatic genes, a final iteration of expression profiling was performed on two different single-cell progenies of PC3-ML cells, namely PC3-ML clone 1 (PC3-ML1) and clone 3 (PC3-ML3), with demonstrated metastatic bone-tropism in the animal model. This more stringent analysis, using genetically homogeneous cell populations, showed that when compared to PC3-N cells, PC3-ML1 and PC3-ML3 cells showed differential regulation of 261 genes and 100 genes, respectively, and 37 genes were found to overlap between these two clonal cell populations (FIG. 5). A comparative analysis of these 37 genes with the 6 genes identified above resulted in a set of 3 upregulated genes, IL1B, CXCL6 and PI3, which consistently correlated with either PDGFRs expression and the bone-metastatic potential of all the human prostate cancer cell lines that were tested in the study (FIG. 5).

Proteins encoded by these three genes present functional characteristics that are highly suggestive of their implication in neoplastic transformation and progression. Without wishing to be limited by theory, the three-gene set identified above defines at least one malignant phenotype emerging in androgen-deprived prostate cancer patients, in which the acquisition of neuroendocrine features by cancer cells is associated with bone-tropism and metastatic progression.

All three gene products are secreted proteins involved in inflammation, recruitment and activation of immune cells and in part bone metabolism and turnover. Without wishing to be limited by theory, their combined pro-metastatic effect could involve cross talking with the surrounding stromal cells, thus rendering the marrow a compatible microenvironment for metastatic progression.

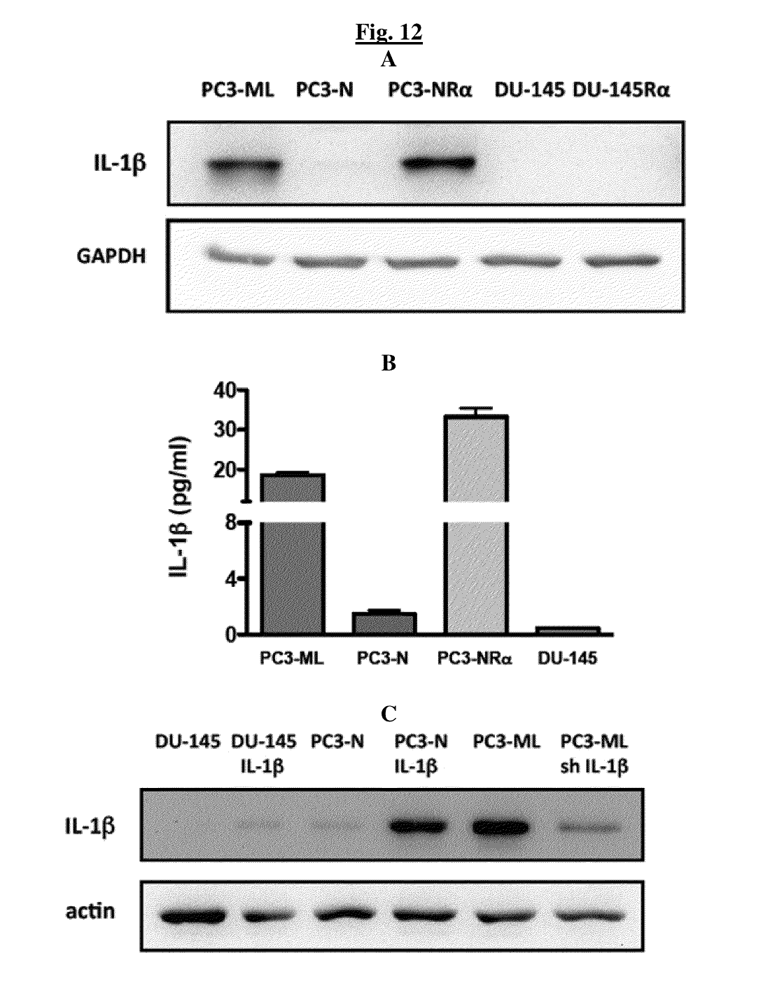

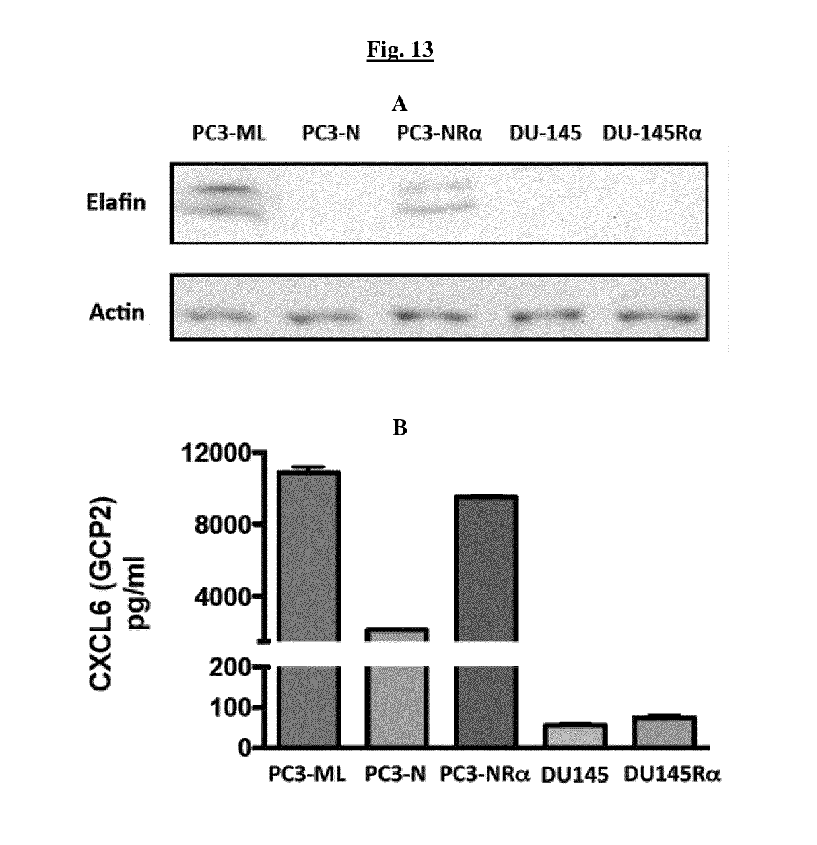

Proteomic approaches validated the transcriptome analysis and confirmed the data relative to IL-1β (FIG. 12) as well as CXCL6 and Elafin (FIG. 13).

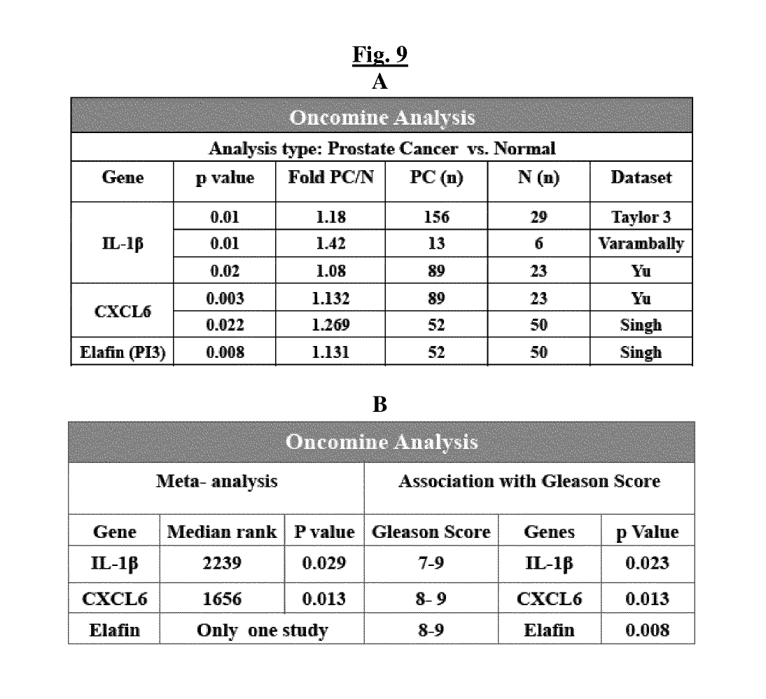

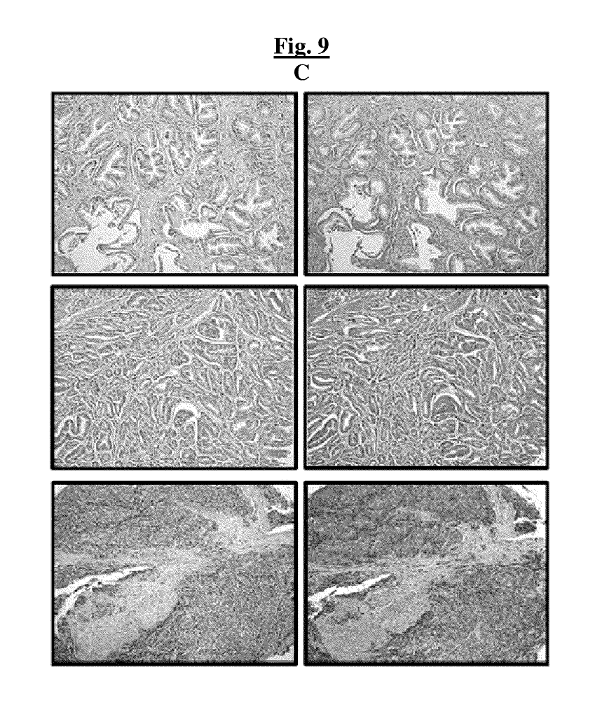

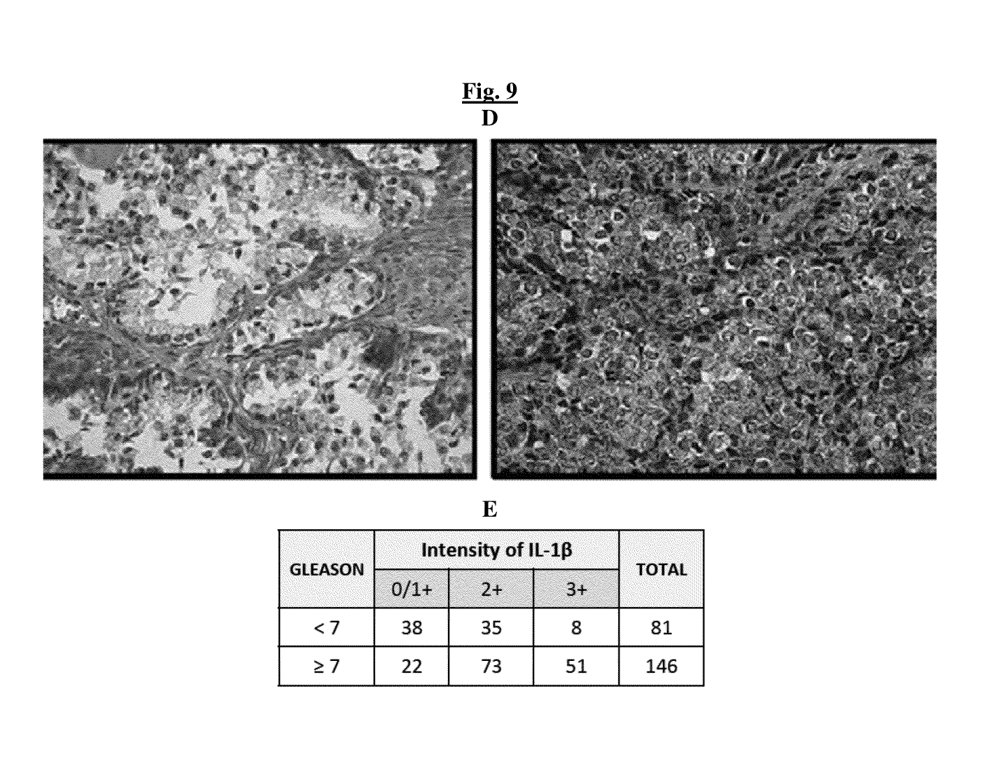

These results were corroborated by mining prostate cancer data sets publically available through the Oncomine repository, showing that IL-1β, CXCL6, and Elafin are significantly upregulated in tumor as compared to normal prostate tissues (FIG. 9A). Furthermore, a meta-analysis indicated a strong association of both IL-1β and CXCL6 with prostate cancer with Gleason scores (≧7) (FIG. 9B). In light of these observations, human tissue arrays were screened including 227 cases of prostate adenocarcinoma for IL-1β protein expression and correlated signal intensities with the Gleason score attributed to each tissue specimen (FIGS. 9C-9D). This approach validated the results from the Oncomine analysis and conclusively demonstrates that prostate tumors with intermediate and high Gleason scores, which have the highest propensity to metastasize (Bastian et al., 2006, Cancer 107:1265-72; Yigitbasi et al., 2011, Urol. Oncol. 29:162-5), express increased levels of IL-1β as compared to tumors with Gleason scores (<7) or normal tissues (FIG. 9E). Remarkably, IL-1β inhibits the expression of both PSA (Abdul & Hoosein, 2000. Cancer Lett. 149:37-42) and AR (Culig et al., Endocr. Relat. Cancer. 9:155-70) in prostate cancer cells, thus reproducing features observed in PC3-ML cells as well as NEPC cells either primarily or as a consequence of ADT. In one embodiment. IL-1β is an important player in the establishment of skeletal secondary lesions by prostate cancer.

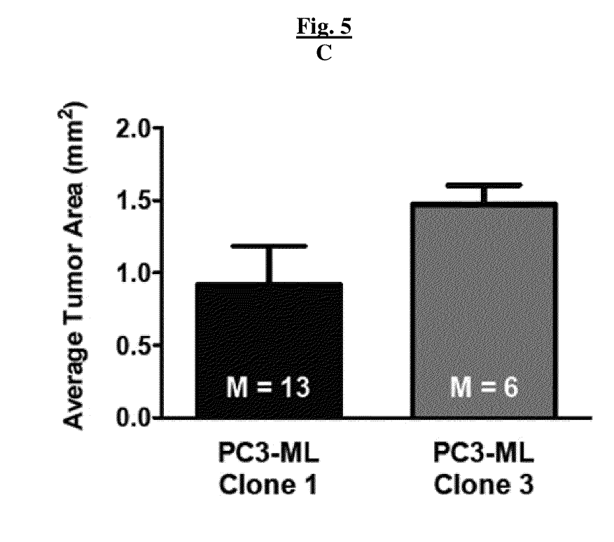

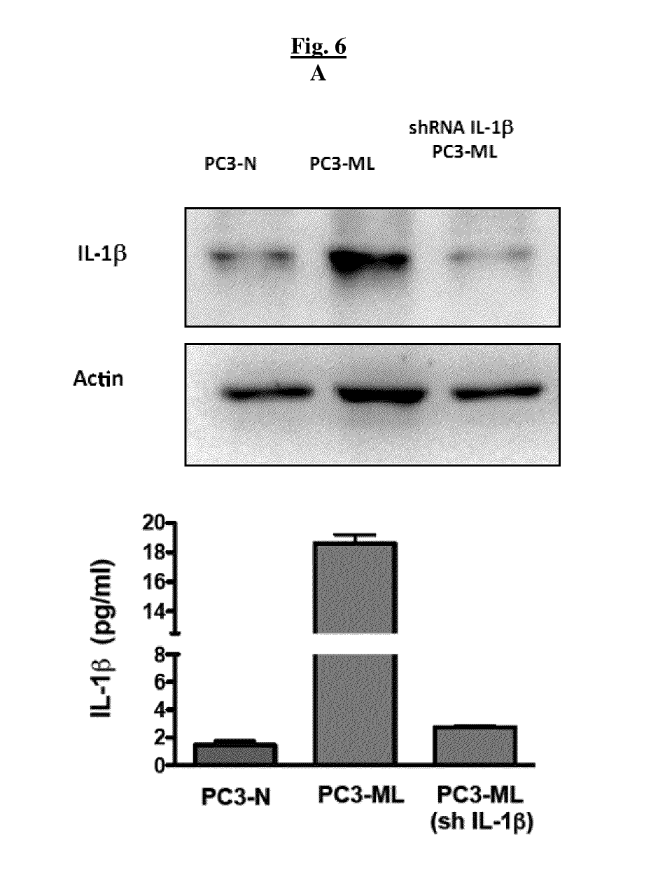

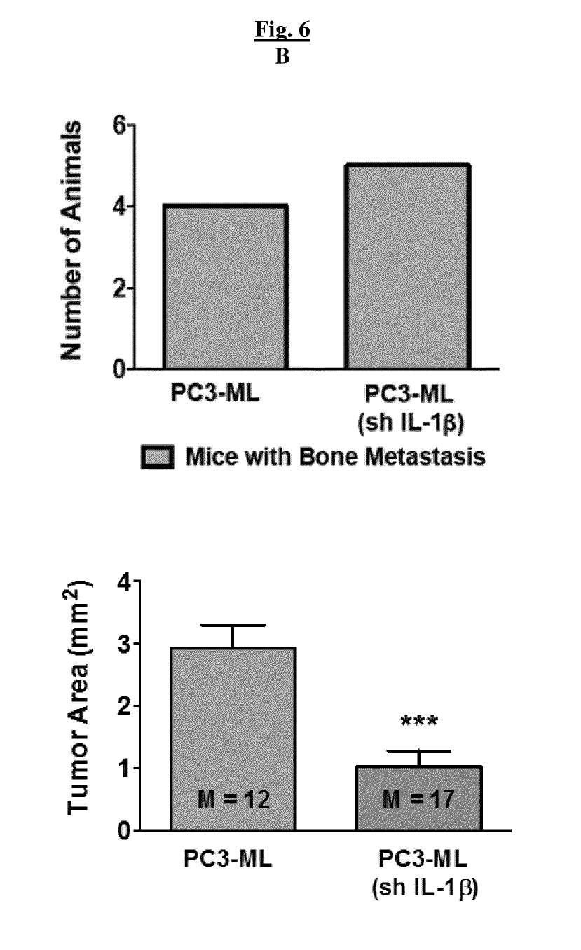

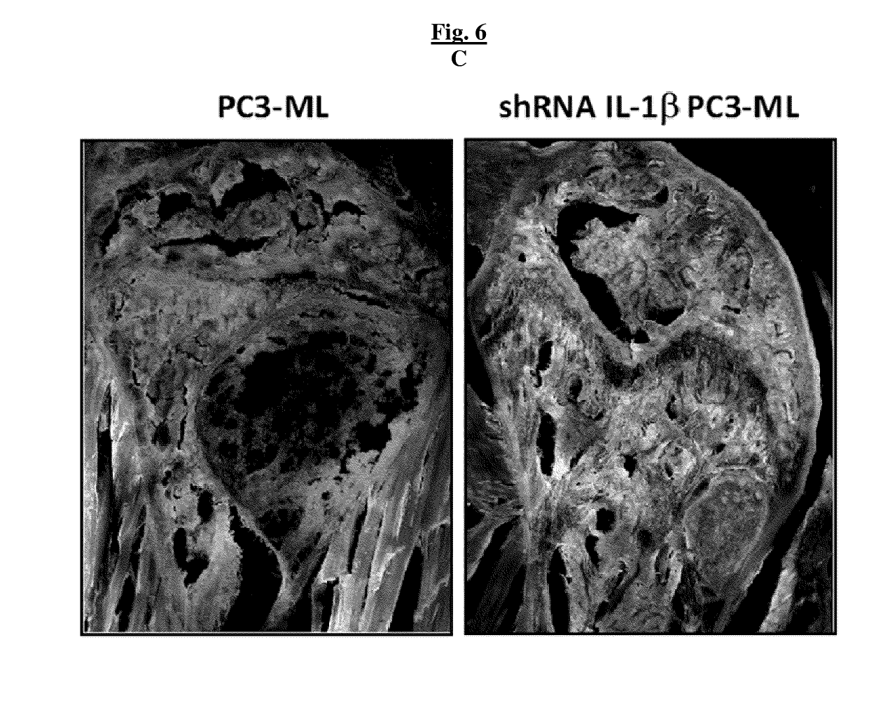

To test this hypothesis, RNAi depletion of IL-1β was used in PC3-ML bone-metastatic prostate cancer cells, in light of the regulatory role that this cytokine exerts on both CXCL6 and Elafin. The silencing by stable transduction of shRNA decreased IL-1β in PC3-ML cells to expression levels lower than PC3-N cells (FIG. 6A) and also reduced the size of bone-metastatic tumors by approximately 70% in mice inoculated with these cells via the systemic arterial circulation four weeks before (FIGS. 6B-6C). In one embodiment, silencing IL-1β in combination with one or both of the other two genes identified in this study provides superior inhibition of metastatic progression than silencing IL-1β alone.

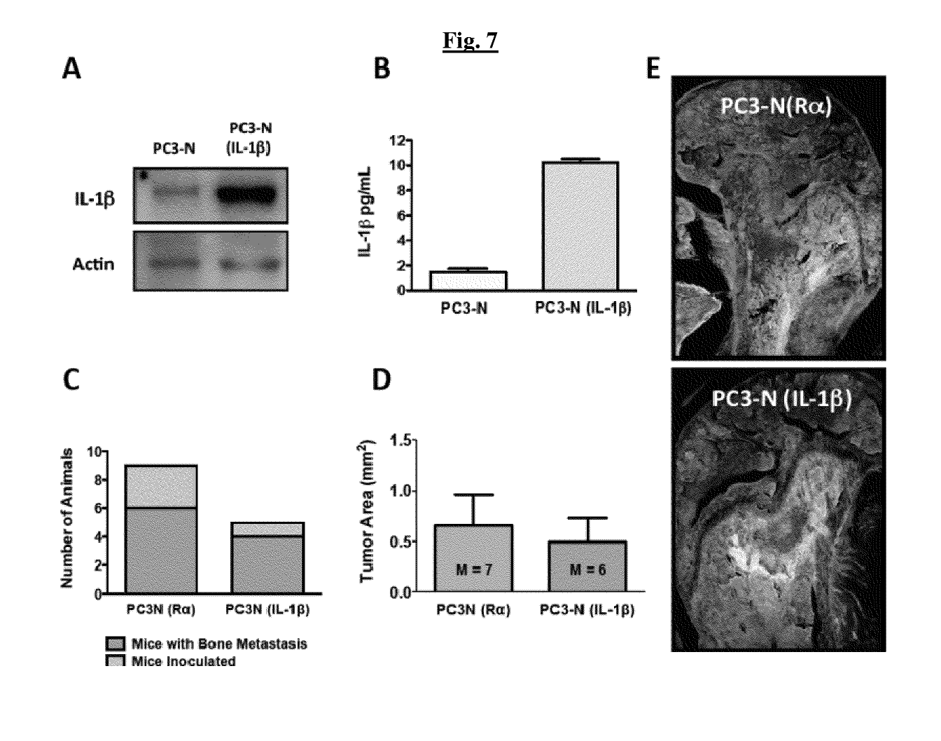

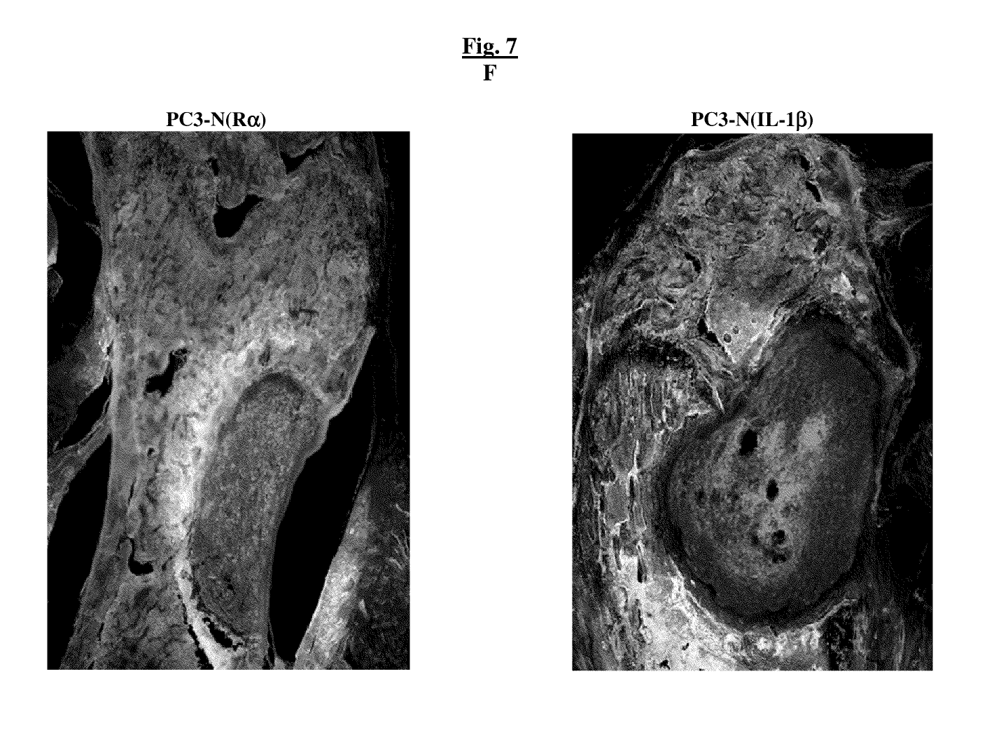

In another set of experiments, IL-1β was exogenously overexpressed to ascertain whether it could promote metastases in prostate phenotypes normally unable to progress from DTCs to macroscopic lesions in the bone marrow. First and in contrast to PC3-N cells, which survive maximum three weeks as small skeletal foci and only in 20% of the inoculated animals (Russell et al., 2009, Oncogene 28:412-421), PC3-N(IL-1β) cells were able to generate metastatic lesions that could be detected at four weeks post-inoculation and measured in number and size comparably to those produced by PC3-N(Rα) cells (FIGS. 7A-7D).

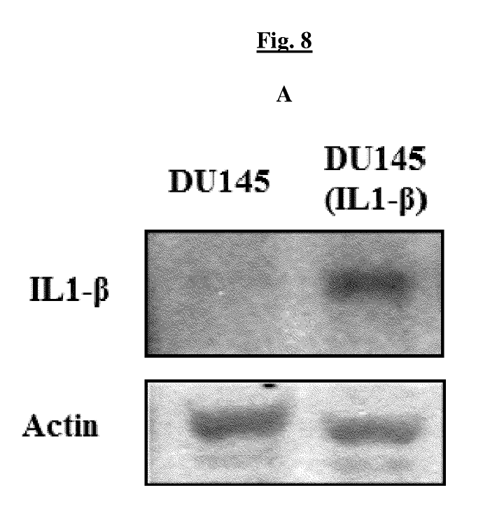

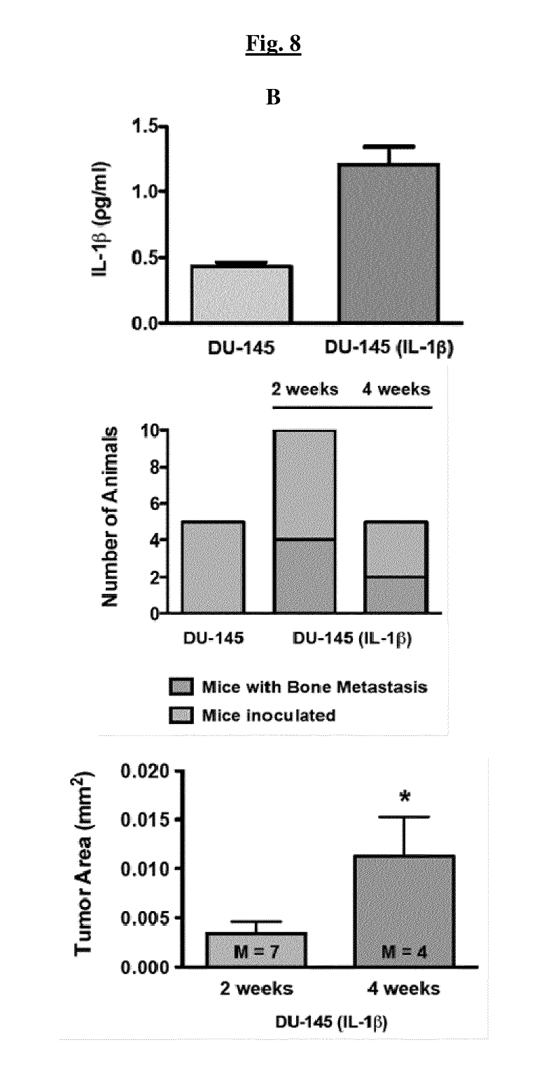

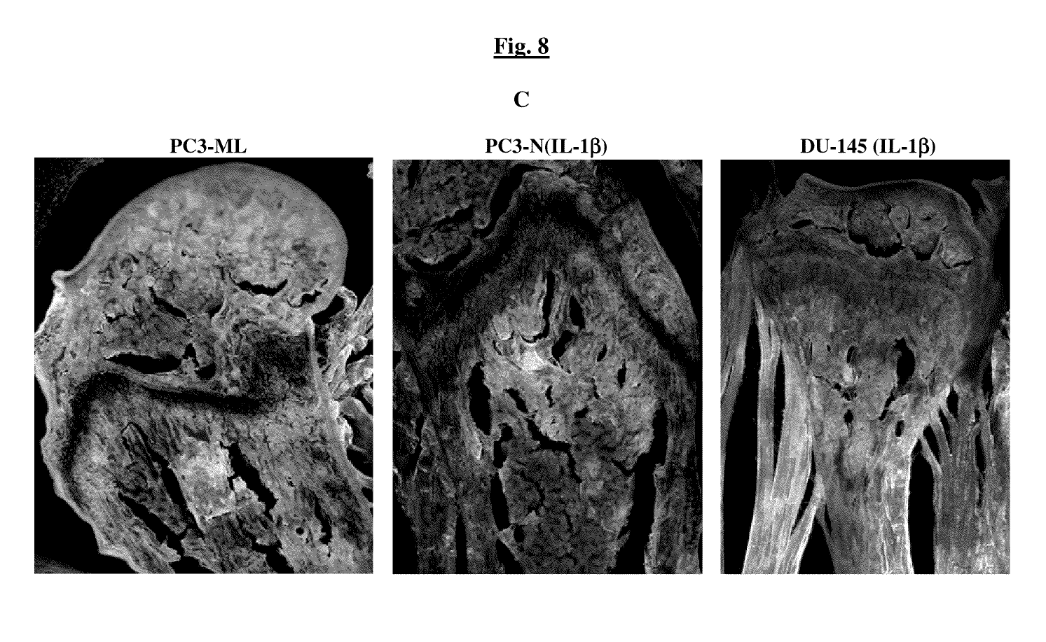

Even more compelling results were obtained with DU-145 cells. DU-145 cells do not endogenously express IL-1β, and can survive as isolated DTCs for no longer than three days in the skeleton of inoculated animals. Upon stable overexpression of IL-1β (FIG. 8A) generated bone tumors that were detected at two weeks post-inoculation in 40% of mice (FIG. 8C-). Although these metastases were smaller in size than similar lesions produced by either PC3-ML or PC3-N(IL-1β) cells after the same time interval, the study provided the first evidence that IL-1β induces a bone-metastatic phenotype even in malignant prostate cells that were originally derived from a brain secondary lesion and notoriously lack skeletal tropism when tested in mouse models.

Furthermore, the increase in tumor area observed for DU-145 (IL-1β) lesions detected four weeks compared to those at two weeks post-inoculation (FIG. 8C) clearly indicates that IL-1β promotes both survival and proliferation of metastatic cells in the skeleton.

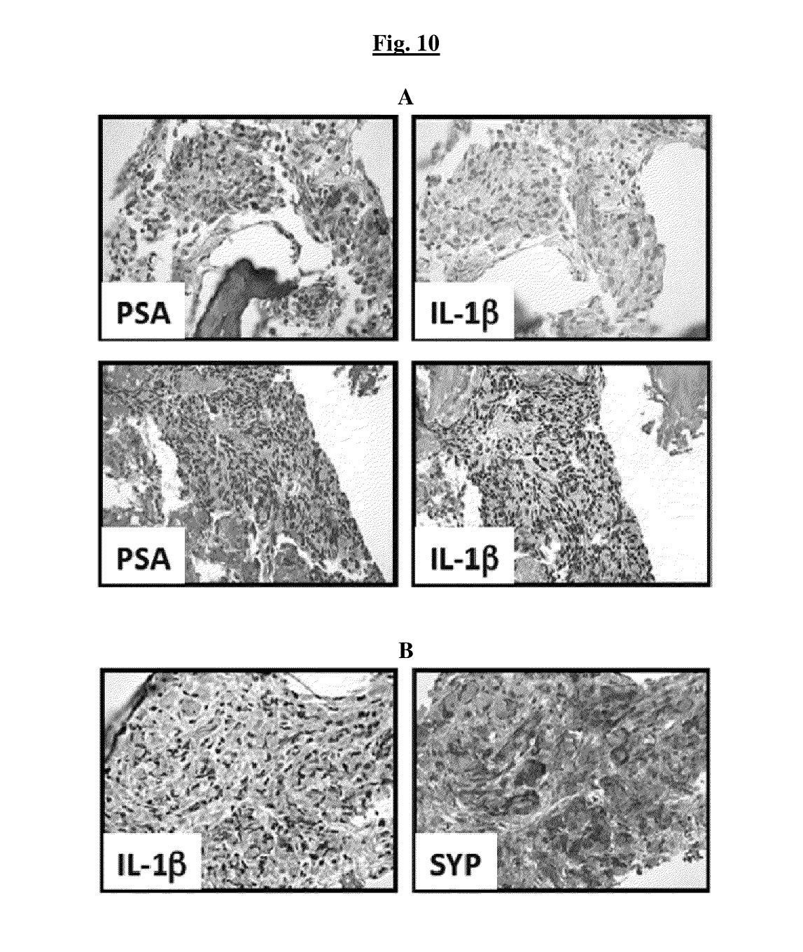

Despite originating from different metastatic sites in prostate cancer patients. PC3 and DU-145 cells both express neuroendocrine markers (Leiblich et al., 2007, Prostate 67:1761-9). The acquisition of a neuroendocrine phenotype is frequently induced by the ADT commonly employed for patients with advanced prostate adenocarcinoma (Miyoshi et al., 2001, BJU Int. 88:982-3; Frigo & McDonnell, 2008, Mol. Cancer Ther. 7:659-69) and is also observed in transgenic animal models of prostate cancer upon castration (Huss et al., 2007, Neoplasia. 9:938-50). In one embodiment, the convergence of ADT-induccd neuroendocrine transdifferentiation and increased expression of IL-1β underpins the propensity of prostate cancer cells to colonize the skeleton and eventually progress to secondary bone lesions. An ex-vivo analysis of skeletal lesions obtained from patients with advanced prostate cancer showed that all specimens stained positively for IL-1β. Interestingly, the intensity of the signal showed an inverse correlation with PSA expression (FIG. 10A), and two specimens also stained positively for synaptophysin (FIG. 10B). These results provide strong support for a role of IL-1β over-expression in bonemetastatic growth of prostate cancer cells and suggest a frequent association of this cytokine with evident NEPC features of skeletal metastases from prostate adenocarcinoma.

The secondary tropism of metastatic tumors is the result of compatibility between DTCs and the tissue microenvironment of the colonized organs (Shibue & Weinberg, 2011, Semin. Cancer Biol. 21:99-106). Because of the stimulatory effect exerted by IL-1β on the bone-resorption activity of osteoclasts, a plausible scenario would include this cytokine supporting secondary skeletal lesions by promoting bone matrix turnover and increasing the availability of trophic factors for the disseminated cancer cells. This mechanism is targeted by bisphosphonates and RANKL inhibitors in the clinical management of metastatic breast and prostate cancer patients. Since osteoclasts are not involved in these early phases of bone marrow colonization, the pro-metastatic role of IL-1β identified here is exerted in one embodiment through either autocrine trophic stimulation of cancer cells, or in another embodiment a more complex paracrine recruitment of surrounding bone stromal cells other than osteoclasts. In the latter scenario. IL-1β stimulates cells of the bone stroma and induces them to reciprocate with increased or ex novo production of trophic factors, which support the survival and growth of DTCs.

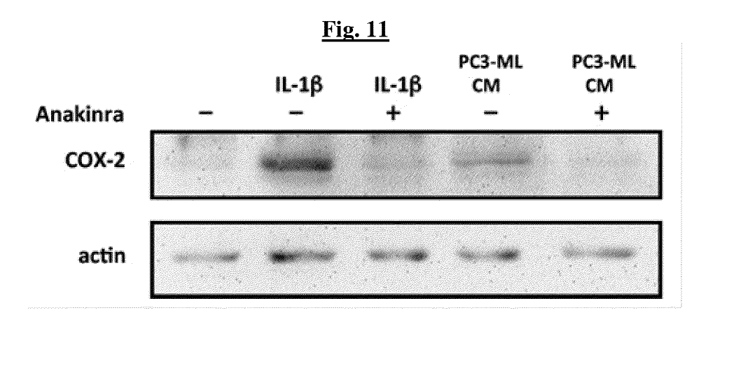

In order to investigate the pro-metastatic role of IL-1β, human MSCs were exposed to media conditioned by PC3-ML cells and the effects on COX-2 expression was measured after 48 hours. The increase in COX-2 observed in MSCs was induced by the IL-1β secreted by PC3-ML cells, since the IL-1R antagonist Anakinra was able to completely prevent it (FIG. 11).

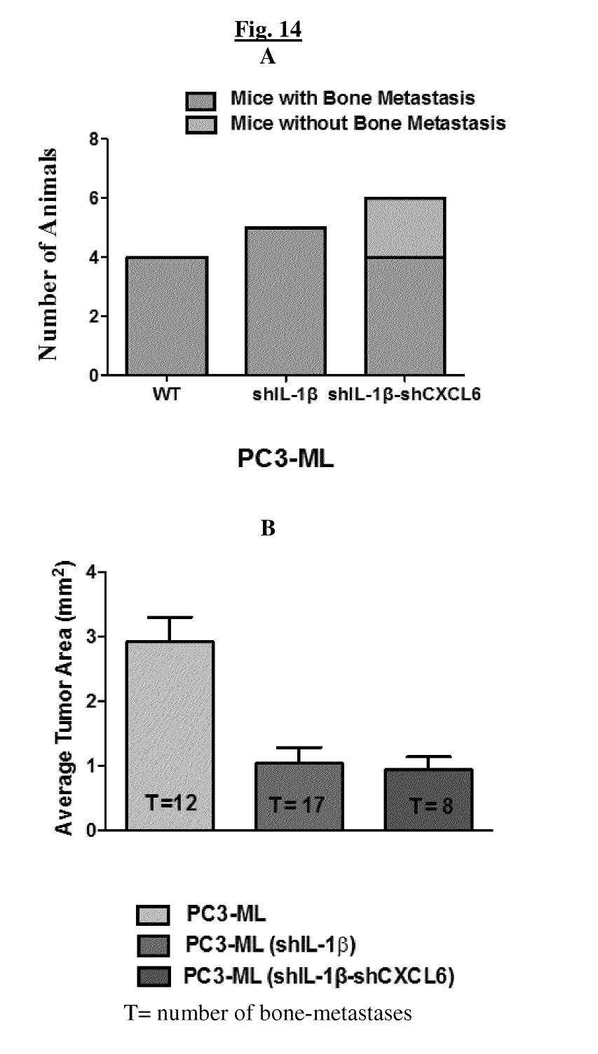

FIG. 14, comprising FIGS. 14A-14B, illustrates the results of in vivo experiments conducted using human prostate cancer cells double-KO for IL-1β and CXCL6. In these experiments, as illustrated in FIG. 14, 30% of inoculated mice were metastasis-free and the remaining animals showed fewer bone tumors as compared to controls or IL-1beta KO mice. These observations strongly support the important role of both molecules in the bone-metastatic behavior of prostate cancer. Further, this experiment supports the combined therapeutic targeting of IL-1β and CXCL6 in the treatment of metastatic bone cancer associated with primary prostate or breast cancers.

The present studies highlight the importance of early survival of DTCs mediated by IL-1β for successful lodging and initial colonization of the bone. These events are particularly relevant for the seeding of additional tumors by Circulating Tumor Cells (CTCs) dislodged from pre-existing lesions and commonly detected in the peripheral blood of metastatic patients. Thus, disrupting the functional interactions between IL-1β and its receptors, most likely IL-1R, substantially attenuates the progression of prostate cancer at the skeletal level and possibly reduces the secondary involvement of other organs.

Notably, therapeutics that target either IL-1β or IL-1R are currently available and prescribed for skeletal inflammatory conditions of non-neoplastic etiology such as rheumatoid arthritis. The evidence provided by the present study leads to the repositioning of these drugs in the clinic and rapidly translate into novel strategies for treating existing metastatic skeletal lesions as well as preventing ongoing seeding of additional lesions both in bone and visceral organs.

### Antibodies

The invention contemplates using a composition comprising an antibody selected from the group consisting of an IL-1β antibody, a CXCL6 antibody, an Elafin antibody and any combination thereof within the methods of the invention. In another embodiment, the antibody is selected from the group consisting of XOMA-052, AMG-108, canakinumab, rilonacept, LY-2189102, and any combinations thereof. In one embodiment, the antibody comprises an antibody selected from a polyclonal antibody, a monoclonal antibody, a humanized antibody, a synthetic antibody, a heavy chain antibody, a human antibody, and a biologically active fragment of an antibody.

It will be appreciated by one skilled in the art that an antibody comprises any immunoglobulin molecule, whether derived from natural sources or from recombinant sources, which is able to specifically bind to an epitope present on a target molecule. In one embodiment, the target molecule comprises IL-1β, CXCL6 or Elafin.

In one aspect of the invention, the target molecule is directly neutralized by an antibody that specifically binds to an epitope on the target molecule. In another aspect of the invention, the effects of the target molecule are blocked by an antibody that specifically binds to an epitope on a downstream effector. In still another aspect of the invention, the effects of the target molecule are blocked by an antibody that binds to an epitope of an upstream regulator of the target molecule.

When the antibody to the target molecule used in the compositions and methods of the invention is a polyclonal antibody (IgG), the antibody is generated by inoculating a suitable animal with a peptide comprising full length target protein, or a fragment thereof, an upstream regulator, or fragments thereof. These polypeptides, or fragments thereof, may be obtained by any methods known in the art, including chemical synthesis and biological synthesis, as described elsewhere herein. In this regard, an exemplary IL-1β sequence is SEQ ID NO:1, an exemplary CXCL6 sequence is SEQ ID NO:3, and an exemplary Elafin sequence is SEQ ID NO:4. Antibodies produced in the inoculated animal that specifically bind to the target molecule, or fragments thereof, are then isolated from fluid obtained from the animal.

Antibodies may be generated in this manner in several non-human mammals such as, but not limited to goat, sheep, horse, camel, rabbit, and donkey. Methods for generating polyclonal antibodies are well known in the art and are described, for example in Harlow et al., 1998, In: Antibodies, A Laboratory Manual, Cold Spring Harbor, N.Y.

Monoclonal antibodies directed against a full length target molecule, or fragments thereof, may be prepared using any well-known monoclonal antibody preparation procedures, such as those described, for example, in Harlow et al. (1998, In: Antibodies, A Laboratory Manual, Cold Spring Harbor, NY) and in Tuszynski et al. (1988, Blood, 72:109-115). Human monoclonal antibodies may be prepared by the method described in U.S. Patent Publication No. 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al., 1992, Critical Rev. Immunol. 12(3,4):125-168, and the references cited therein.

When the antibody used in the methods of the invention is a biologically active antibody fragment or a synthetic antibody corresponding to antibody to a full length target molecule, or fragments thereof, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology available in the art, and described, for example, in Wright et al., 1992, Critical Rev. in Immunol, 12(3,4):125-168 and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen et al. (U.S. Pat. No. 6,180,370), Wright et al., 1992, Critical Rev. Immunol. 12(3,4):125-16$, and in the references cited therein, or in Gu et al., 1997, Thrombosis & Hematocyst 77(4):755-759, or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to humanized immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well-known procedures. Preferably, the human constant region DNA sequences are isolated from immortalized B-cells as described in International Patent Application Publication No. WO 1987/02671. CDRs useful in producing the antibodies of the present invention may be similarly derived from DNA encoding monoclonal antibodies capable of binding to the target molecule. Such humanized antibodies may be generated using well-known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al. 1998, Antibodies: A Laboratory Manual, Cold Spring Harbor, N.Y.

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al., 1989, Nature 341:544-546 (incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as target molecule depletors in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase, cDNA that specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al, 2001, Molecular Cloning: A Laboratory Manual, Cold Spring Harbor Laboratory Press. Cold Spring Harbor, N.Y.

Bacteriophage that encode the desired antibody may be engineered such that the protein is displayed on the surface thereof in such a manner that it is available for binding to its corresponding binding protein, e.g., the antigen against which the antibody is directed. Thus, when bacteriophage that express a specific antibody are incubated in the presence of a cell which expresses the corresponding antigen, the bacteriophage will bind to the cell. Bacteriophage that do not express the antibody will not bind to the cell. Such panning techniques are well known in the art and are described for example, in Wright et al., 1992, Critical Rev. Immunol. 12(3,4):125-168.

Processes such as those described above, have been developed for the production of human antibodies using M13 bacteriophage display (Burton et al., 1994. Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage that display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995. J. Mol. Biol. 248:97-105).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used to practice the method of the invention, or to prepare a pharmaceutical composition useful in practicing the method of the invention.

The antibodies of the present invention can be assayed for immunospecific binding by any method known in the art. The immunoassays that can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g. Current Protocols in Molecular Biology. (Ausubel et al., eds.), Greene Publishing Associates and Wiley-Interscience, New York (2002)). Exemplary immunoassays are described briefly below (but are not intended to be in any way limiting).

### Methods of the Invention

The invention includes a method of treating metastasis in a subject diagnosed with cancer. The method comprises determining whether at least one gene encoding a protein selected from the group consisting of IL-1β, CXCL6, Elafin and any combination thereof is upregulated in a cancer tissue sample from the subject as compared to the level of expression of the at least one gene in a non-cancer control sample of the same tissue. According to the method, if the at least one gone is upregulated in the cancer tissue sample from the subject, the subject is administered a therapeutically effective amount of a pharmaceutical composition comprising a IL-1β-depleting agent and at least one therapeutic agent comprising a CXCL6-depleting agent or an Elafin-depleting agent.

The invention further includes a method of preventing metastasis in a subject diagnosed with cancer. The method comprises determining whether at least one gene encoding a protein selected from the group consisting of IL-1β, CXCL6. Elafin and any combination thereof is upregulated in a cancer tissue sample from the subject as compared to the level of expression of the at least one gene in a non-cancer control sample of the same tissue. According to the method, if the at least one gene is upregulated in the cancer tissue sample from the subject, the subject is administered a therapeutically effective amount of a pharmaceutical composition comprising a IL-1β-depleting agent and at least one therapeutic agent comprising a CXCL6-depleting agent or an Elafin-depleting agent.

In one embodiment, the cancer comprises a solid cancer. In another embodiment, the solid cancer is selected from the group consisting of breast cancer and prostate cancer. In yet another embodiment, the metastasis comprises bone metastasis. In yet another embodiment, the at least one gene encodes IL-1β. In yet another embodiment, the at least one gene encodes IL-1β, CXCL6 and Elafin. In yet another embodiment, the at least one gene encodes IL-1β, CXCL6 and Elafin. In yet another embodiment, the at least one gene is upregulated by at least 50% as compared to the level of expression of the at least one gene in a non-cancer control sample of the same tissue.

In one embodiment, the IL-1β-depleting agent is selected from the group consisting of anakinra, XOMA-052, AMG-108, canakinumab, rilonacept, K-832, CYT-013-IL bQb, LY-2189102, dexamethasone, interferon-gamma, pentoxifylline, an IL-1β antibody, siRNA, ribozyme, an antisense, an aptamer, a peptidomimetic, a small molecule, and a combination thereof. In another embodiment, the IL-1β antibody comprises an antibody selected from the group comprising a polyclonal antibody, monoclonal antibody, humanized antibody, synthetic antibody, heavy chain antibody, human antibody, biologically active fragment of an antibody, and any combination thereof. In yet another embodiment, the CXCL6-depleting agent is a CXCL6 antibody selected from the group consisting of a polyclonal antibody, monoclonal antibody, humanized antibody, synthetic antibody, heavy chain antibody, human antibody, biologically active fragment of an antibody, and any combination thereof. In yet another embodiment, the Elafin-depleting agent is an Elafin antibody selected from the group consisting of a polyclonal antibody, monoclonal antibody, humanized antibody, synthetic antibody, heavy chain antibody, human antibody, biologically active fragment of an antibody, and any combination thereof.

In one embodiment, the method further comprises administering to the subject an additional agent selected from the group consisting of a chemotherapeutic agent, an anti-cell proliferation agent and any combination thereof. In another embodiment, the chemotherapeutic agent comprises an alkylating agent, nitrosourea, antimetabolite, antitumor antibiotic, plant alkyloid, taxane, hormonal agent, bleomycin, hydroxyurea, L-asparaginase, or procarbazine. In yet another embodiment, the anti-cell proliferation agent comprises granzyme, a Bcl-2 family member, cytochrome C, or a caspase. In yet another embodiment, the at least one therapeutic agent and the additional agent are co-administered to the subject. In yet another embodiment, the at least one therapeutic agent and the additional agent are co-formulated and co-administered to the subject. In yet another embodiment, the pharmaceutical composition is administered to the subject by an administration route selected from the group consisting of inhalational, oral, rectal, vaginal, parenteral, topical, transdermal, pulmonary, intranasal, buccal, ophthalmic, intrathecal, and any combination thereof. In yet another embodiment, the subject is a mammal. In yet another embodiment, the mammal is a human.

### Kits of the Invention

The invention includes a kit comprising a IL-1β-depleting agent and at least one therapeutic agent comprising a CXCL6-depleting agent or an Elafin-depleting agent, an applicator, and an instructional material for use thereof. The instructional material included in the kit comprises instructions for preventing or treating metastasis in a subject diagnosed with cancer. The instructional material recites that the level of expression of at least one gene encoding a protein selected from the group consisting of IL-1β, CXCL6, Elafin and any combination thereof in a cancer tissue sample from the subject is compared to the levels of expression of the at least one gene in a non-cancer control sample of the same tissue. The instructional material further recites that, if the at least one gene is upregulated in the cancer tissue sample from the subject, the subject is administered a therapeutically effective amount of a pharmaceutical composition comprising the IL-1β-depleting agent and the at least one therapeutic agent comprising a CXCL6-depleting agent or an Elafin-depleting agent.

In one embodiment, the cancer comprises ovarian cancer or prostate cancer. In another embodiment, the metastasis comprises bone metastasis. In yet another embodiment, the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, an IL-antibody, siRNA, ribozyme, an antisense, an aptamer, a peptidomimetic, a small molecule, and any combination thereof. In yet another embodiment, the CXCL6-depleting agent is a CXCL6 antibody selected from the group consisting of a polyclonal antibody, monoclonal antibody, humanized antibody, synthetic antibody, heavy chain antibody, human antibody, biologically active fragment of an antibody, and any combination thereof. In yet another embodiment, the Elafin-depleting agent is an Elafin antibody selected from the group consisting of a polyclonal antibody, monoclonal antibody, humanized antibody, synthetic antibody, heavy chain antibody, human antibody, biologically active fragment of an antibody, and any combination thereof.

### Combination Therapies

The compounds identified using the methods described here are useful in the methods of the invention in combination with at least one additional compound useful for treating cancer. This additional compound may comprise compounds identified herein or compounds, e.g., commercially available compounds, known to treat, prevent, or reduce the symptoms of cancer and/or metastasis.

In one aspect, the present invention contemplates that the agents useful within the invention may be used in combination with a therapeutic agent such as an anti-tumor agent, including but not limited to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include dactinomycin, daunorubicin, doxorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine that are also useful in the invention.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-α, gelonin, Prodigiosin, a ribosome-inhibiting protein (RIP), Pseudommoas exotoxin, Clostridium difficile Toxin B, Helicobacter priori VacA, Yersinia enterocolitica YopT. Violacein, diethylenetriaminepentaacetic acid, irofulven, Diptheria Toxin, mitogillin, ricin, botulinum toxin, cholera toxin, saporin 6, or a combination thereof.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Pharmaceutical Compositions and Formulations

The invention envisions the use of a pharmaceutical composition comprising an IL-1β-depleting agent and at least one therapeutic agent comprising a CXCL6-depleting agent or an Elafin-depleting agent, within the methods of the invention.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions are formulated using one or more pharmaceutically acceptable excipients or carriers. In one embodiment, the pharmaceutical compositions comprise a therapeutically effective amount of at least IL-1β-depleting agent and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 1991, Mack Publication Co., New Jersey.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one that comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

### Administration/Dosing

The regimen of administration may affect what constitutes an effective amount. For example, the therapeutic formulations may be administered to the patient either prior to or after a surgical intervention related to cancer, or shortly after the patient was diagnosed with cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat cancer in the patient. An effective amount of the therapeutic compound necessary to achieve a therapeutic effect may vary according to factors such as the activity of the particular compound employed; the time of administration; the rate of excretion of the compound; the duration of the treatment; other drug, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound cant be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 7,500 mg; about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of cancer in a patient.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing cancer in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically- or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form. For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation. In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours. The term pulsatile release is used herein in its conventional sense to refer to a drug formulation that provides release of the drug in such a way as to produce pulsed plasma profiles of the drug after drug administration. The term immediate release is used in its conventional sense to refer to a drug formulation that provides for release of the drug immediately after drug administration.

As used herein, short-term refers to any period of time up to and including about 8 hours, about 7 hours, about 6 hour, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods:

### Cell Lines and Cell Culture

NIH-3T3 and DU-145 cells were obtained from ATCC, and passaged in the laboratory for less than 6 months after resuscitation. PC3-N and PC3-ML sub-lines were derived from the parental PC-3 cell line. Both sub-lines were tested by Idexx Radii (Columbia, Mo.) by STR-based DNA finger printing and confirmed to be of human origin without mammalian inter-species contamination. The alleles for 9 different markers were determined and the genetic profiles of both PC3-ML and PC3-N cell were found identical to the profiles reported for the parental PC3 line deposited with the ATCC.

Cells lines were cultured at 37° C. and 5% CO₂ in DMEM supplemented with 10% fetal bovine serum and 0.1% gentamicin. All prostate cancer cell lines were cultured for ten passages and then thawed a new frozen stock to avoid the emergence of genotypic and phenotypic changes.

Cells were genetically engineered to stably-express EGFP using a lentiviral vector (AmeriPharma), with or without the coding sequence for either PDGFRα or a retroviral vector containing the coding sequence PDGFRβ or IL1-β (Origen).

Bone marrow-derived Human Mesenchymal Stem cells (MSCs) (Lonza, Allendale, N.J.) were used between passage 5 and 8 and cultured in MSC growth medium (α-MEM (Invitrogen) supplemented with 10% FBS, 1 ng/ml bFGF (R&D Systems, Minneapolis, Minn.), and 0.1% gentamicin).

### Overexpression of PDGFRβ or IL1-β in Cells

To prepare PDGFR β or IL1-β overexpression retrovirus, a mixture of pLXSN vector or pLXSN containing the PDGFRβ or IL1-β (50 ng) plasmid and 8 μl of lipofectamine 2000 were mixed and incubated at room temperature for 30 min. The transfection mix was transferred to Phoenix cells that were approximately 70% confluent. After 16 hours the media were replaced with growth medium with 10% serum, and virus was harvested 38 hours post-transfection.

The viral harvest was repeated twice every 24 hours. The virus-containing medium was pooled, centrifuged at 44,000 rpm for 20 minutes, and the supernatant was used to infect PC3-N, DU 145 and 22RVI cells. The successfully infected cells were selected for the ability to proliferate in media containing G418 (0.6 mg/ml). The resulting cells were characterized by Western blot analysis using an antibody against PDGFRβ or IL1-β (Santa Cruz).

### Suppression of IL1-β Expression in PC3-ML Cells

Mission TRC lentiviral shRNA vectors (Sigma-Aldrich) were used to knock down IL1-β. The following shRNA sequence was used:

(SEQ ID NO: 2)
5′-CGGCCAGGATATAACTGACTT-3′.

Lentiviral particles were used to infect sub-continent cell cultures, overnight in the presence of 8 μg/ml polybrene (Millipore). The successful infected cells were selected for the ability to proliferate in media containing G418 (0.6 μg/ml). The resulting cells were characterized by Western blot analysis using an antibody against IL1β.

### Immunoprecipitation and Western Blot

Cells (80% confluence) were cultured in DMEM without serum for 4 hours, and then treated with PDGF-B (30 ng/ml) or human bone marrow (1:20) for 15 minutes. The cells were washed twice with ice-cold phosphate buffered saline (PBS), and then lysed in cell lysis buffer (25 nM Tris-HCL, 150 mM NaCl, 5 mM NaF, 1 mM EDTA, 1% Igepal, 1% Phosphatase inhibitor cocktail set II, and 1% Protease inhibitor cocktail set III (Calbiochem).

Lysates were clarified by centrifugation at 14,000 rpm, 4° C. for 15 minutes, and PDGFRβ was immunoprecipitated from the lysates as previously described. The antibody for immunoprecipitation of PDGFRβ was a rabbit polyclonal anti-PDGFRβ (Santa Cruz). Membranes were blotted with antibodies against phospho-tyrosine (Cell signaling technology) and PDGFRβ (Santa Cruz).

Cell lysates were obtained and SDS-PAGE and Western Blot analysis performed as previously described, with few modifications. Membranes were blotted with antibodies targeting PDGFRα, PDGFRβ, IL1-β (SC-7884, Santa Cruz, Dallas, Tex.), Actin (A-2066, Sigma-Aldrich, St. Louis, Mo.), COX-2 (ab15191, AbCam, Cambridge, UK) Elafin (SC-20637, Santa Cruz) and GAPDH (DI6H11, Cell Signaling Technology, Beverly, Mass.). Primary antibody binding was detected using an HRP-conjugated secondary antibody (Pierce). Chemiluminescence signals were obtained using SuperSignal West Femto reagents (Pierce) and detected with the Fluorochem 8900 imaging system and relative software (Alpha Innotech).

### RT-PCR Analysis

RNA was collected from cell lysates using the Qiagen RNeasy Mini Kit. RT-PCR was run using 1 μg RNA/sample with the Qiagen One Step RT PCR kit and primers for human IL1-β and human GAPDH (Integrated DNA Technologies).

### Animal Model of Metastasis

Five week-old male immunocompromised mice (CBI 7-SCRF) were obtained from Taconic and housed in a germ-free barrier. At six weeks of age, mice were anesthetized with 100 mg/kg ketamine and 20 mg/kg xylazine and successively inoculated in the left cardiac ventricle with cancer cells (5×10⁴ in 100 μl of serum-free DMEM/F12). Cell inoculation was performed using a 30-gauge needle connected to a 1 ml syringe. The delivery of the cell suspension in the systemic blood circulation was validated by the co-injection of blue-fluorescent 10 μm polystyrene beads (Invitrogen-Molecular Probes). Animals were randomly assigned to different experimental groups and sacrificed at specified time points following inoculation. Organs were harvested and prepared as described below and tissue sections inspected blindly for metastatic lesions. The homogeneous and numerically-consistent distribution of the beads in adrenal glands and lungs collected at necropsy and inspected by fluorescence microscopy were used as discrimination criteria for the inclusion of animals in the studies.

All experiments were conducted in accordance with NIH guidelines for the humane use of animals. All protocols involving the use of animals were approved by the Drexel University College of Medicine Committee for the Use and Care of Animals.

### Tissue Preparation

Bones and soft-tissue organs were collected and fixed in 4% formaldehyde solution for 24 hours and then transferred into fresh formaldehyde for additional 24 hours. Soft tissues were then placed either in 30% sucrose for cryoprotection or 1% formaldehyde for long-term storage. Bones were decalcified in 0.5 M EDTA for 7 days followed by incubation in 30% sucrose. Tissues were maintained at 4° C. for all aforementioned steps and frozen in O.C.T. medium by placement over dry-ice chilled 2-methylbutane. Serial sections of 80 μm in thickness were obtained using a Microm HM550 cryostat.

### Fluorescence Stereomicroscopy and Morphometric Analysis of Metastases

Bright-field and fluorescent images of skeletal metastases were acquired using an Olympus stereomicroscope coupled to an Olympus DT70 CCD color camera. Digital images were analyzed with ImageJ software and calibrated for measurement by obtaining a pixel to millimeter ratio. A freehand tool was used to outline the border of each metastatic lesion, and the area was computed using the ImageJ ‘measure area’ function.

### Conditioned Media Experiments

Conditioned media were obtained according to Li et al., 2012, Cancer Disc. 2:840-55. In brief, 7.5×10⁵ PC3-ML cells were plated in 15 ml of DMEM supplemented with 10% fetal bovine serum and 0.1% gentamicin and cultured for five days. The medium from each dish was then collected and centrifuged at 2000 rpm for 10 minutes and then used fresh as described below.

For bone cell-treatment experiments, MSCs were plated at least 48 hours prior to treatment; when 70% confluent, cells were incubated in a 1:1 mixture of conditioned medium and MSC growth medium for 48 hours. To pharmacologically induce the over-expression of COX-2 in MSCs, cells were exposed to 0.1 ng/ml of IL-1β (R&D Systems). Cells were pre-incubated with the IL-1β inhibitor Anakinra (Amgen. Chesterbrook, Pa.) at a 10 μg/ml concentration for 30 minutes prior to being exposed to IL-1α.

### ELISA Measurements

The concentrations of IL-1β and CXCL6 were measured by ELISA as described in the manufacturers' protocols. In brief, 5×10⁵ cells were plated in a 35 mm² dish; the next day the medium was replaced with 1 ml of DMEM supplemented with 10% fetal bovine serum and 0.1% gentamicin and cultured for 24 hours. The supernatant was then collected and the protein concentration was measured using Quantikine kits (R&D systems).

### Example 1

### Microarray Analysis

Total RNA of each sample was collected from a 60 mm-dish and was purified with Qiagen RNeasy Mini kit (according to the manufactory manual). RNA qualities for samples were checkered by BioAnalyzer (Agilent. Palo Alto, Calif.) before proceeding to further investigations. Two rounds of amplification were employed according to Affymetrix Two-cycle Amplification protocol using 25 ng for total RNA of collected cells. 15 μg aliquots of amplified biotinylated RNA were hybridized to 1.0 Human Gene ST array (Affymetrix). Arrays were scanned using the GencChip Scanner 3000 (Affymetrix). GeneSpring software version 11.5 was used to filter and complete the statistical analysis.

To analyze the microarray data, CEL files were loaded to the GeneSpring 11.5 software and probeset summarization was conducted using the RMA 16 algorithm. For each probe, the median of the log summarized values from all the samples was calculated and subtracted from each sample. After processing and normalization, the resulting 28,869 genes included in the 1.0 Human Gene ST arrays were then filtered to remove very low or saturated signal values. Each entity was filtered on raw data filter by percentile with an upper cut off of 100 and a lower cut off 20. The resulting new entities were then subjected to statistical analysis using unpaired T-Test with a p-value fixed at 0.05. Finally, a higher-stringency filter was applied to the resulting entities using a fold change cut-off of 2.0 and Benjamin-Hochberg multiple testing corrections. The microarray data were submitted to the Gene Expression Omnibus (GEO) data repository and can be accessed with the number GSE43332.

| | | | | Feld Change Cut-off 2.0 |
|---|---|---|---|---|
| | | Filter Transcripts by | | and BH Multiple Testing |
| Cell lines | All Entities | Expression Value | Statistical Analysis | Correction |
| PC3-ML vs. PC3-N | 28869 | 22955 | 1522 | 16 |
| PC3-N(FDGFRα) vs. PC3-N | 28869 | 22980 | 2173 | 40 |
| DU145(FDGFRα) vs. DU145 | 28869 | 22884 | 1403 | [symbol] |
| PC3-N(FDGFRβ) vs. PC3-N | 28869 | 22972 | 2101 | 71 |
| PC3-ML(Clone 1) vs. PC3-N | 28869 | 23044 | 4 [symbol] 46 | 2 [symbol] 1 |
| PC3-ML(Clone 3) vs. PC3-N | 28869 | 23015 | 3036 | 100 |

[symbol]

indicates data missing or illegible when filed

In order to identify genes associated with metastasis phenotype, Venn diagrams were employed to capture commonalties between bone metastasis cell lines (FIGS. 3B-3C, 4D and 5).

### Example 2

### Oncomine Analysis

The Oncomine database was searched for IL-1β, CXCL6, and PI3 genes. The data sets containing expression data for each gene were filtered to display upregulation in prostate cancer versus normal prostate tissue with p<0.05. If more than one data set passed the filters, a meta-analysis was performed to obtain a p value.

### Example 3

### Clinical Samples, Immunohistochemistry and Analysis

Commercially available human Tissue Microarrays (TMAs, PR956, PR8010, PR483, PR751) contained 192 prostate tissue cores and were obtained from US Biomax (Rockville, Md.). Two additional existing TMAs containing 35 de-identified human prostate cancer specimens as well as seven de-identified bone tissue specimens with metastatic prostate cancer were obtained from the archives of the Department of Pathology at Drexel University College of Medicine.

Immunohistochemical detection was conducted using antibodies against IL-1β (ab2105, AbCam), PSA (ER-PR8, Cell Marque) and Synaptophysin (SP11, Ventana, Oro Valley, Ariz.) all diluted 1:50 on formalin-fixed paraffin-embedded sections. The staining conditions using the BenchMark ULTRA IHC/ISH Staining module were as follows: antigen retrieval (pH 8.1) using CC1 reagent 64 minutes, followed by primary antibody incubation for 40 minutes at 37° C. and then staining with the XT, Ultraview™ Universal DAB Detection Kit. Interpretation and scoring was conducted by two clinical pathologists (F.U.G. and M.I.L.) using light microscopy. Staining intensities were scored as follows 0: no staining, 1: weak staining, 2: moderate staining and 3: strong staining. Only samples that showed ≧40% of cellular staining were used for the analysis.

The number and size of skeletal metastases between two experimental groups were analyzed using a two-tailed Student's t-test and between multiple groups using a one-way ANOVA test. A value of p<0.05 was deemed significant. The results of TMA staining were subjected to chi-square analysis and plotted in a contingency table.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of treating or preventing metastasis in a subject diagnosed with cancer, the method comprising:
determining whether at least one gene encoding a protein selected from the group consisting of IL-1β, CXCL6, Elafin and any combination thereof is upregulated in a cancer tissue sample from the subject as compared to the level of expression of the at least one gene in a non-cancer control sample of the same tissue, and,
if the at least one gene is upregulated in the cancer tissue sample from the subject, administering to the subject a therapeutically effective amount of a pharmaceutical composition comprising a IL-1β-depleting agent and at least one therapeutic agent comprising a CXCL6-depleting agent or an Elafin-depleting agent, whereby the metastasis of the subject diagnosed with cancer is treated or prevented.

2. The method of claim 1, wherein the cancer comprises a solid cancer.

3. The method of claim 2, wherein the solid cancer is selected from the group consisting of breast cancer and prostate cancer.

4. The method of claim 1, wherein the metastasis comprises bone metastasis.

5. The method of claim 1, wherein the at least one gene encodes IL-1β.

6. The method of claim 1, wherein the at least one gene is upregulated by at least 50% as compared to the level of expression of the at least one gene in a non-cancer control sample of the same tissue.

7. The method of claim 1, wherein the IL-1β-depleting agent is selected from the group consisting of anakinra, XOMA-052, AMG-108, canakinumab, rilonacept, K-832, CYT-013-IL1bQb, LY-2189102, dexamethasone, interferon-gamma, pentoxifylline, an IL-1β antibody, siRNA, ribozyme, an antisense, an aptamer, a peptidomimetic, a small molecule, and a combination thereof.

8. The method of claim 7, wherein the IL-1β antibody comprises an antibody selected from the group comprising a polyclonal antibody, monoclonal antibody, humanized antibody, synthetic antibody, heavy chain antibody, human antibody, biologically active fragment of an antibody, and any combination thereof.

9. The method of claim 1, wherein the CXCL6-depleting agent is a CXCL6 antibody selected from the group consisting of a polyclonal antibody, monoclonal antibody, humanized antibody, synthetic antibody, heavy chain antibody, human antibody, biologically active fragment of an antibody, and any combination thereof.

10. The method of claim 1, wherein the Elafin-depleting agent is an Elafin antibody selected from the group consisting of a polyclonal antibody, monoclonal antibody, humanized antibody, synthetic antibody, heavy chain antibody, human antibody, biologically active fragment of an antibody, and any combination thereof.

11. The method of claim 1, wherein the at least one therapeutic agent comprises a CXCL6-depleting agent.

12. The method of claim 1, wherein the at least one therapeutic agent comprises a CXCL6-depleting agent and an Elafin-depleting agent.

13. The method of claim 1, comprising further administering to the subject an additional compound selected from the group consisting of a chemotherapeutic agent, an anti-cell proliferation agent and any combination thereof.

14. The method of claim 13, wherein the chemotherapeutic agent comprises an alkylating agent, nitrosourea, antimetabolite, antitumor antibiotic, plant alkyloid, taxane, hormonal agent, bleomycin, hydroxyurea, L-asparaginase, or procarbazine.

15. The method of claim 13, wherein the anti-cell proliferation agent comprises granzyme, a Bcl-2 family member, cytochrome C, or a caspase.

16. The method of claim 13, wherein the pharmaceutical composition and the additional compound are co-administered to the subject.

17. The method of claim 14, wherein the pharmaceutical composition and the additional compound are co-formulated and co-administered to the subject.

18. The method of claim 1, wherein the pharmaceutical composition is administered to the subject by an administration route selected from the group consisting of inhalational, oral, rectal, vaginal, parenteral, topical, transdermal, pulmonary, intranasal, buccal, ophthalmic, intrathecal, and any combinations thereof.

19. The method of claim 1, wherein the subject is a mammal.

20. The method of claim 19, wherein the mammal is a human.

21. A kit comprising a IL-1β-depleting agent and at least one therapeutic agent comprising a CXCL6-depleting agent or an Elafin-depleting agent, an applicator, and an instructional material for use thereof,
wherein the instructional material comprises instructions for preventing or treating metastasis in a subject diagnosed with cancer,
wherein the instructional material recites that the level of expression of at least one gene encoding a protein selected from the group consisting of IL-1β, CXCL6, Elafin and any combination thereof in a cancer tissue sample from the subject is compared to the levels of expression of the at least one gene in a non-cancer control sample of the same tissue,
wherein the instructional material further recites that, if the at least one gene is upregulated in the cancer tissue sample from the subject, the subject is administered a therapeutically effective amount of a pharmaceutical composition comprising the IL-1β-depleting agent and the at least one therapeutic agent comprising a CXCL6-depleting agent or an Elafin-depleting agent.

22. The kit of claim 21, wherein the cancer comprises ovarian cancer or prostate cancer.

23. The kit of claim 21, wherein the metastasis comprises bone metastasis.

24. The kit of claim 21, wherein the subject is a mammal.

25. The kit of claim 24, wherein the mammal is human.


SEQUENCE LISTING (4 sequences)

SEQ ID NO: 1 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 2 - DNA, 21 bp, Artificial Sequence
  Chemically Synthesized
cggccaggatataactgactt

SEQ ID NO: 3 - protein, 114 aa, Homo sapiens
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLKKVIQKILDSGNKKN

SEQ ID NO: 4 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ